(19) United States
(12) Patent Application Publication
(10) Pub. No.: US 2012/0237564 A1
(43) Pub. Date: Sep. 20, 2012

(54) Use of Aerosolized Antibiotics for Treating Chronic Obstructive Pulmonary Disease

(75) Inventors: Michael N. Dudley, San Diego, CA (US); Neil Berkley, San Diego, CA (US); David C. Griffith, San Marcos, CA (US); Jeffery S. Loutit, Los Altos, CA (US); Elizabeth E. Morgan, Escondido, CA (US); Keith A. Bostian, Atherton, CA (US); Sanjay Sethi, East Amherst, NY (US)

(73) Assignee: MPEX Pharmaceuticals, Inc., San Diego, CA (US)

(21) Appl. No.: 13/398,310

(22) Filed: Feb. 16, 2012

Related U.S. Application Data
(63) Continuation of application No. PCT/US2010/002307, filed on Aug. 19, 2010.
(60) Provisional application No. 61/235,319, filed on Aug. 19, 2009.
(60) Provisional application No. 61/240,749, filed on Sep. 9, 2009.
(60) Provisional application No. 61/249,228, filed on Oct. 6, 2009.

(51) Int. Cl. A61K 31/5383; A61K 9/14; A61P 11/08; A61K 31/56; A61P 11/00; A61P 31/04

(52) U.S. Cl. 424/400; 514/230.2; 514/171

(57) ABSTRACT

The present invention relates to methods and compositions for treating obstructive pulmonary disorders. In particular, compositions and methods described herein relate to the use of an aerosolized antibiotic for treating obstructive pulmonary disorders including chronic obstructive pulmonary disorder (COPD) and chronic bronchitis (CB).

### RELATED APPLICATIONS

This application is a continuation of International Patent Application No. PCT/US2010/002307 filed Aug. 19, 2010 which claims the benefit of priority under 35 U.S.C. §119 to U.S. Provisional Application No. 61/235,319, filed Aug. 19, 2009, U.S. Provisional Application No. 61/240,749, filed Sep. 9, 2009, and U.S. Provisional Application No. 61/249,228, filed Oct. 6, 2009, each entitled “USE OF AEROSOLIZED LEVOFLOXACIN FOR TREATING CHRONIC OBSTRUCTIVE PULMONARY DISEASE,” which are hereby incorporated by reference in their entireties.

### FIELD OF THE INVENTION

The present invention relates to methods and compositions for treating obstructive pulmonary disorders. In particular, compositions and methods described herein relate to the use of aerosolized antibiotics, such as, levofloxacin or ofloxacin, for treating obstructive pulmonary disorders including chronic obstructive pulmonary disorder (COPD) and chronic bronchitis (CB).

### BACKGROUND

Chronic obstructive pulmonary disease (COPD) is a heterogeneous group of slowly progressive diseases characterized by airflow obstruction that interferes with normal breathing (Rennard S I. COPD: overview of definitions, epidemiology, and factors influencing its development. Chest 1998; 113 (Suppl 4):235-41s). COPD includes disorders such as emphysema, chronic bronchitis (CB), and asthma. Approximately one in 20 deaths in the United States has COPD as the underlying cause.

An acute exacerbation of COPD is a sudden worsening of COPD symptoms, such as shortness of breath, increased cough, quantity and color of phlegm. Periodic exacerbations in COPD are associated with enormous healthcare expenditures and significant morbidity, specifically an increased risk of death, a decline in pulmonary function, and a significant change in quality of life. There is a need for therapies that reduce acute exacerbations and resulting hospitalization.

### SUMMARY

The present invention relates to methods and compositions for treating obstructive pulmonary disorders in subjects. Obstructive pulmonary disorders can include chronic obstructive pulmonary disorder (COPD) and chronic bronchitis (CB). Some methods and compositions relate to reducing the incidence and/or severity of an acute exacerbation of an obstructive pulmonary disorder. Some methods and compositions relate to preventing an acute exacerbation of an obstructive pulmonary disorder. Some embodiments relate to preventing or the incidence and/or duration of hospitalization of a subject.

Acute exacerbations of an obstructive pulmonary disorder may be indicated by symptoms that can include increased sputum production, more purulent sputum, change in sputum color, increased coughing, increased wheezing, chest tightness, reduced exercise tolerance, increased fatigue, fluid retention, acute confusion, worsened dyspnea, and combinations thereof.

Some embodiments include methods for treating a pulmonary disorder selected from chronic obstructive pulmonary disease (COPD) and chronic bronchitis (CB), in which the methods includes administering to a human in need thereof an aerosolized antibiotic, such as an aerosolized solution comprising levofloxacin or ofloxacin and a divalent or trivalent cation, wherein the period between acute exacerbations is increased.

Some embodiments include methods for reducing the rate of acute exacerbations of a pulmonary disorder selected from chronic obstructive pulmonary disease (COPD) and chronic bronchitis (CB) in a population of humans having said disorder. Some such embodiments include administering to said population an aerosol of a solution comprising levofloxacin or ofloxacin. In some such embodiments, the rate of exacerbations in said population is reduced to less than about 6 exacerbations/patient year, less than about 2.9 exacerbations/patient year, less than about 1.4 exacerbations/patient year, less than about 1.3 exacerbations/patient year, less than about 1.2 exacerbations/patient year, less than about 1.1 exacerbations/patient year, and less than about 0.5 exacerbations/patient year.

Some embodiments include the use of an aerosol of a solution comprising levofloxacin or ofloxacin for reducing the rate of acute exacerbations of a pulmonary disorder selected from chronic obstructive pulmonary disease (COPD) and chronic bronchitis (CB) in a population of humans having said disorder. In some such embodiments, the rate of exacerbations in said population is reduced to less than about 2.9 exacerbations/patient year, less than about 1.4 exacerbations/patient year, less than about 1.3 exacerbations/patient year, less than about 1.2 exacerbations/patient year, and less than about 1.1 exacerbations/patient year.

Some embodiments include methods for increasing the period of time between acute exacerbations of a pulmonary disorder selected from chronic obstructive pulmonary disease (COPD) and chronic bronchitis (CB) in a population of humans having said disorder. Some such methods include administering to said population an aerosol of a solution comprising levofloxacin or ofloxacin. In some such embodiments, the period of time between acute exacerbations for the median population is increased to greater than about 287 days, greater than about 281 days, greater than about 200 days, greater than about 100 days, and greater than about 50 days.

Some embodiments include the use of an aerosol of a solution comprising levofloxacin or ofloxacin for increasing the period of time between acute exacerbations of a pulmonary disorder selected from chronic obstructive pulmonary disease (COPD) and chronic bronchitis (CB) in a population of humans having said disorder. In some such embodiments, the period of time between acute exacerbations for the median population is increased to greater than about 287 days, greater than about 281 days, greater than about 200 days, greater than about 100 days, and greater than about 50 days.

Some embodiments include methods for reducing the rate of respiratory-related hospitalizations in a population of humans having a disorder selected from chronic obstructive pulmonary disease (COPD) and chronic bronchitis (CB). Some such methods include administering to said population an aerosol of a solution comprising levofloxacin or ofloxacin. In some such embodiments, the rate of respiratory-related hospitalizations in said population is reduced to less than about 0.45 respiratory-related hospitalizations/patient year, less than about 0.35 respiratory-related hospitalizations/patient year, less than about 0.42 exacerbation-related hospitalization/patient year, less than about 0.27 exacerbation-related hospitalization/patient year.

Some embodiments include the use of an aerosol of a solution comprising levofloxacin or for reducing the rate of respiratory-related hospitalizations in a population of humans having a disorder selected from chronic obstructive pulmonary disease (COPD) and chronic bronchitis (CB). In some such embodiments, the rate of respiratory-related hospitalizations in said population is reduced to less than about 0.45 respiratory-related hospitalizations/patient year, less than about 0.35 respiratory-related hospitalizations/patient year, less than about 0.42 exacerbation-related hospitalization/patient year, less than about 0.27 exacerbation-related hospitalization/patient year.

In some of the foregoing embodiments, the solution further comprises a divalent or trivalent cation.

In some of the foregoing embodiments, the solution consists essentially of levofloxacin or ofloxacin and a divalent or trivalent cation.

In some of the foregoing embodiments, the solution comprises no lactose.

In some of the foregoing embodiments, the solution comprises a divalent or trivalent cation concentration from about 50 mM to about 400 mM, and a levofloxacin or ofloxacin concentration from between about 50 mg/ml to about 200 mg/ml.

In some of the foregoing embodiments, the solution comprises a divalent or trivalent cation concentration from about 100 mM to about 300 mM, and a levofloxacin or ofloxacin concentration from between about 75 mg/ml to about 150 mg/ml.

In some of the foregoing embodiments, the solution comprises a divalent or trivalent cation concentration from about 150 mM to about 250 mM, and a levofloxacin or ofloxacin concentration from between about 90 mg/ml to about 125 mg/ml.

In some of the foregoing embodiments, the solution comprises an osmolality from about 300 mOsmol/kg to about 500 mOsmol/kg, and a pH from about 5 to about 8.

The method of any one of claims 1, 7, and 14, or use of any one of claims 2, 8 and 15, wherein the solution comprises an osmolality from about 350 mOsmol/kg to about 425 mOsmol/kg, and a pH from about 5 to about 6.5.

In some of the foregoing embodiments, the solution comprises a pH from about 5.5 to about 6.5.

In some of the foregoing embodiments, the solution comprises a divalent or trivalent cation selected from magnesium, calcium, zinc, copper, aluminum, and iron.

In some of the foregoing embodiments, the solution comprises magnesium chloride.

In some of the foregoing embodiments, the solution comprises a levofloxacin or ofloxacin concentration between about 90 mg/ml to about 110 mg/ml, a magnesium chloride concentration between about 175 mM to about 225 mM, a pH between about 5 to about 7; an osmolarity of between about 300 mOsmol/kg to about 500 mOsmol/kg, and lacks lactose.

In some of the foregoing embodiments, the aerosol comprises a mass median aerodynamic diameter from about 2 microns to about 5 microns with a geometric standard deviation less than or equal to about 2.5 microns.

In some of the foregoing embodiments, the aerosol comprises a mass median aerodynamic diameter from about 2.5 microns to about 4.5 microns with a geometric standard deviation less than or equal to about 1.8 microns.

In some of the foregoing embodiments, the aerosol comprises a mass median aerodynamic diameter from about 2.8 microns to about 4.3 microns with a geometric standard deviation less than or equal to about 2 microns.

In some of the foregoing embodiments, the aerosol is produced with a vibrating mesh nebulizer. In some such embodiments, the vibrating mesh nebulizer is a PART E-FLOW® nebulizer.

In some of the foregoing embodiments, at least about 20 mg of levofloxacin or ofloxacin is administered to the lungs of the human, at least about 100 mg of levofloxacin or ofloxacin is administered to the lungs of the human, at least about 125 mg of levofloxacin or ofloxacin is administered to the lungs of the human, and at least about 150 mg of levofloxacin or ofloxacin is administered to the lungs of the human.

In some of the foregoing embodiments, the aerosol is administered to the lungs of the human in less than about 10 minutes, in less than about 5 minutes, in less than about 3 minutes, and in less than about 2 minutes.

Some of the foregoing embodiments also include co-administering an additional active agent selected from the group consisting of antibiotics, bronchodilators, anticholinergics, glucocorticoids, eicosanoid inhibitors, and combinations thereof. In some such embodiments, co-administering comprises inhaling the additional active agent. In more such embodiments, the antibiotic is selected from the group consisting of tobramycin, aztreonam, ciprofloxacin, azithromycin, tetracycline, quinupristin, linezolid, vancomycin, and chloramphenicol, colisitin and combinations thereof. In more such embodiments, the bronchodilator is selected from the group consisting of salbutamol, levosalbuterol, terbutaline, fenoterol, terbutlaine, pirbuterol, procaterol, bitolterol, rimiterol, carbuterol, tulobuterol, reproterol, salmeterol, formoterol, arformoterol, bambuterol, clenbuterol, indacterol, theophylline, roflumilast, cilomilast, and combinations thereof. In more such embodiments, the anticholinergic is selected from the group consisting of ipratropium, tiotropium, and combinations thereof. In more such embodiments, the glucocorticoid is selected from the group consisting of prednisone, fluticasone, budesonide, mometasone, ciclesonide, beclomethasone, and combinations thereof. In more such embodiments, the eicosanoid is selected from the group consisting of montelukast, pranlukast, zafirlukast, zileuton, ramatroban, seratrodast, and combinations thereof.

Some of the foregoing embodiments comprise administering the aerosol once daily.

Some of the foregoing embodiments comprise administering the aerosol twice daily.

### DETAILED DESCRIPTION

The present invention relates to methods and compositions for treating obstructive pulmonary disorders. Obstructive pulmonary disorders can include chronic obstructive pulmonary disorder (COPD), chronic bronchitis (CB), and asthma. In some methods, the incidence and severity of acute exacerbations are reduced. In more methods, the acute exacerbations are prevented.

Accordingly, the present invention relates to methods for treating obstructive pulmonary disorders such as COPD and CB. Some methods include administering an aerosol of an antibiotic. In some embodiments, the antibiotic is a fluoroquinolone, such as levofloxacin or ofloxacin, formulated with a divalent or trivalent cation. In some embodiments, the incidence and/or severity of acute exacerbations can be reduced. In some embodiments, the antibiotic is administered to COPD patients at high risk for exacerbations. In some embodiments, the administration decreases the incidence of those acute exacerbations. In some embodiments, the administration lowers the bacterial burden in chronically infected high risk patients and reduces inflammation. In some embodiments, the administration reduces the onset of further infections that may cause acute exacerbations.

In addition to the foregoing, some embodiments include reducing the frequency of acute exacerbations. In some embodiments, the time period between acute exacerbations is increased. In some embodiments, the frequency of hospitalizations is decreased.

### DEFINITIONS

The term “administration” or “administering” refers to a method of giving a dosage of an antimicrobial pharmaceutical composition to a vertebrate. The preferred method of administration can vary depending on various factors, e.g., the components of the pharmaceutical composition, the site of the potential or actual bacterial infection, the microbe involved, and the severity of an actual microbial infection.

A “carrier” or “excipient” is a compound or material used to facilitate administration of the compound, for example, to increase the solubility of the compound. Solid carriers include, e.g., starch, lactose, dicalcium phosphate, sucrose, and kaolin. Liquid carriers include, e.g., sterile water, saline, buffers, non-ionic surfactants, and edible oils such as oil, peanut and sesame oils. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, incorporated by reference herein in its entirety.

A “diagnostic” as used herein is a compound, method, system, or device that assists in the identification and characterization of a health or disease state. The diagnostic can be used in standard assays as is known in the art.

The term “mammal” is used in its usual biological sense. Thus, it specifically includes humans, cattle, horses, dogs, and cats, but also includes many other species.

The term “microbial infection” refers to the undesired proliferation or presence of invasion of pathogenic microbes in a host organism. This includes the excessive growth of microbes that are normally present in or on the body of a mammal or other organism. More generally, a microbial infection can be any situation in which the presence of a microbial population(s) is damaging to a host mammal. Thus, a microbial infection exists when excessive numbers of a microbial population are present in or on a mammal's body, or when the effects of the presence of a microbial population(s) is damaging the cells or other tissue of a mammal.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobioic acid, tartaric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsufuric acid, naphthalene-1,5-disulfonic acid, naphthalene-2-sulfonic acid, and saccharin.

“Solvate” refers to the compound formed by the interaction of a solvent and fluoroquinolone antimicrobial, a metabolite, or salt thereof. Suitable solvates are pharmaceutically acceptable solvates including hydrates.

In the context of the response of a microbe, such as a bacterium, to an antimicrobial agent, the term “susceptibility” refers to the sensitivity of the microbe for the presence of the antimicrobial agent. So, to increase the susceptibility means that the microbe will be inhibited by a lower concentration of the antimicrobial agent in the medium surrounding the microbial cells. This is equivalent to saying that the microbe is more sensitive to the antimicrobial agent. In most cases the minimum inhibitory concentration (MIC) of that antimicrobial agent will have been reduced. The MIC₉₀ can include the concentration to inhibit growth in 90% of organisms.

By “therapeutically effective amount” or “pharmaceutically effective amount” is meant a fluoroquinolone antimicrobial agent, as disclosed for this invention, which has a therapeutic effect. The doses of fluoroquinolone antimicrobial agent which are useful in treatment are therapeutically effective amounts. Thus, as used herein, a therapeutically effective amount means those amounts of fluoroquinolone antimicrobial agent which produce the desired therapeutic effect as judged by clinical trial results and/or model animal infection studies. In particular embodiments, the fluoroquinolone antimicrobial agent are administered in a pre-determined dose, and thus a therapeutically effective amount would be an amount of the dose administered. This amount and the amount of the fluoroquinolone antimicrobial agent can be routinely determined by one of skill in the art, and will vary, depending on several factors, such as the particular microbial strain involved. This amount can further depend upon the patient's height, weight, sex, age and medical history. For prophylactic treatments, a therapeutically effective amount is that amount which would be effective to reduce the onset of the relevant symptom or disorder.

A “therapeutic effect” relieves, to some extent, one or more of the symptoms of the relevant disorder, and includes curing the disorder. “Curing” means that the symptoms of the relevant disorder are eliminated, including the total or substantial elimination of excessive members of viable microbe of those involved in an infection to a point at or below the threshold of detection by traditional measurements. However, certain long-term or permanent effects of a disorder may exist even after a cure is obtained (such as extensive tissue damage). As used herein, a “therapeutic effect” is defined as a statistically significant reduction in a symptom or in bacterial load in a host, emergence of resistance, or improvement in infection symptoms as measured by human clinical results or animal studies.

“Treat,” “treatment,” or “treating,” as used herein refers to administering a pharmaceutical composition for prophylactic and/or therapeutic purposes. The term “prophylactic treatment” refers to treating a patient who does not yet have the relevant disorder, but who is susceptible to, or otherwise at risk of, the disorder such that there is a reduced onset of the disorder. The term “therapeutic treatment” refers to administering treatment to a patient already suffering from the relevant disorder. Thus, in preferred embodiments, treating is the administration to a mammal (either for therapeutic or prophylactic purposes) of therapeutically effective amounts of a fluoroquinolone antimicrobial agent.

Pharmacokinetics (PK) is concerned with the time course of antimicrobial concentration in the body. Pharmacodynamics (PD) is concerned with the relationship between pharmacokinetics and the antimicrobial efficacy in vivo. PK/PD parameters correlate antimicrobial exposure with antimicrobial activity. The rate of killing by antimicrobial is dependent on antimicrobial mode of action and is determined by either the length of time necessary to kill (time-dependent) or the effect of increasing concentrations (concentration-dependent). To predict the therapeutic efficacy of antimicrobials with diverse mechanisms of action different PK/PD parameters may be used. PK/PD parameters may be used to determine the bioavailability of antimicrobial compositions, for example, bioavailability of a composition in the pulmonary system, and/or bioavailability of a composition in plasma/serum.

“AUC/MIC ratio” is one example of a PK/PD parameter. AUC is defined as the area under the plasma/serum or site-of-infection concentration-time curve of an antimicrobial agent in vivo (in animal or human). For example, the site of infection and/or the site where concentration is measured can include portions of the pulmonary system, such as bronchial fluid and/or sputum. Accordingly, AUC may include serum AUC, and pulmonary AUC. AUC₍₀₋ₜ₎ can include the area under curve for time zero to a specific time ‘t.’ AUC_{(0-inf)} can include the area under curve from time zero to infinity. AUC/MIC ratio is determined by dividing the 24-hour-AUC for an individual antimicrobial by the MIC for the same antimicrobial determined in vitro. Activity of antimicrobials with the dose-dependent killing (such as fluoroquinolones) is well predicted by the magnitude of the AUC/MIC ratio.

“Cₘₐₓ:MIC” ratio is another PK:PD parameter. It describes the maximum drug concentration in plasma or tissue relative to the MIC. Fluoroquinolones and aminoglycosides are examples where Cₘₐₓ:MIC may predict in vivo bacterial killing where resistance can be suppressed.

“Time above MIC” (T>MIC) is another PK/PD parameter. It is expressed a percentage of a dosage interval in which the plasma or site-of-infection level exceeds the MIC. Activity of antimicrobials with the time-dependent killing (such as beta-lactams or oxazolidinones) is well predicted by the magnitude of the T>MIC ratio.

The term “dosing interval” refers to the time between administrations of the two sequential doses of a pharmaceutical's during multiple dosing regimens. For example, in the case of orally administered ciprofloxacin, which can be administered twice daily (traditional regimen of 400 mg b.i.d) and orally administered levofloxacin, which can be administered once a day (500 mg or 750 mg q.d.), the dosing intervals are 12 hours and 24 hours, respectively.

As used herein, the “peak period” of a pharmaceutical's in vivo concentration is defined as that time of the pharmaceutical dosing interval when the pharmaceutical concentration is not less than 50% of its maximum plasma or site-of-infection concentration. In some embodiments, “peak period” is used to describe an interval of antimicrobial dosing.

The “respirable delivered dose” is the amount of drug inhaled during the inspiratory phase of the breath simulator that is equal to or less than 5 microns using a simulator programmed to the European Standard pattern of 15 breaths per minute, with an inspiration to expiration ratio of 1:1.

### Methods of Treatment or Prophylaxis

In some embodiments, a method is provided for treating an obstructive pulmonary disorder such as a chronic obstructive pulmonary disorder (COPD), chronic bronchitis (CB), and asthma. In some such embodiments, an animal, specifically including a mammal, suffering from such an obstructive pulmonary disorder, is treated with an antibiotic. In addition, some embodiments include administration of an aerosolized antibiotic for treating an obstructive pulmonary disorder such as a chronic obstructive pulmonary disorder (COPD), chronic bronchitis (CB), and asthma. In some embodiments, the antibiotic is a fluoroquinolone. In some embodiments, the fluoroquinolone is formulated with a divalent or trivalent cation and has improved pulmonary bioavailability

In some methods, the incidence and/or the severity of acute exacerbations are reduced. Acute exacerbations can be indicated by the presence of a symptom that includes increased sputum production, more purulent sputum, change in sputum color, increased coughing, increased wheezing, chest tightness, reduced exercise tolerance, increased fatigue, fluid retention, acute confusion, worsened dyspnea, and combinations thereof. In addition, an acute exacerbation can include a symptomatic respiratory deterioration requiring treatment with antibiotic agents, systemic corticosteroids, hospitalization, or a combination of these treatments.

In some of the above methods, the antibiotic can include quinolones, tetracyclines, glycopeptides, aminoglycosides, β-lactams, rifamycins, macrolides/ketolides, oxazolidinones, coumermycins, chloramphenicol, streptogramins, or polymyxins. In particular embodiments, an antibiotic of the above classes can be, for example, one of the following.

Beta-Lactam Antibiotics

Beta-lactam antibiotics include, but are not limited to, imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefmetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, and LY206763.

Macrolides

Macrolides include, but are not limited to, azithromycin, clarithromycin, erythromycin, oleandomycin, rokitamycin, rosaramicin, roxithromycin, and troleandomycin.

Ketolides

Ketolides include, but are not limited to, telithromycin and cethrimycin.

Quinolones

Quinolones include, but are not limited to, amifloxacin, cinoxacin, ciprofloxacin, enoxacin, fleroxacin, flumequine, lomefloxacin, nalidixic acid, norfloxacin, ofloxacin, levofloxacin, oxolinic acid, pefloxacin, rosoxacin, temafloxacin, tosufloxacin, sparfloxacin, clinafloxacin, moxifloxacin; gemifloxacin; garenofloxacin; PD131628, PD138312, PD140248, Q-35, AM-1155, NM394, T-3761, rufloxacin, OPC-17116, DU-6859a (see, e.g., Sato, K. et al., 1992, Antimicrob Agents Chemother. 37:1491-98), and DV-7751a (see, e.g., Tanaka, M. et al., 1992, Antimicrob. Agents Chemother. 37:2212-18).

Tetracyclines, Glycylcyclines and Oxazolidinones

Tetracyclines, glycylcyclines, and oxazolidinones include, but are not limited to, chlortetracycline, demeclocycline, doxycycline, lymecycline, methacycline, minocycline, oxytetracycline, tetracycline, tigecycline, linezolide, and eperozolid.

Aminoglycosides

Aminoglycosides include, but are not limited to amikacin, arbekacin, butirosin, dibekacin, fortimicins, gentamicin, kanamycin, meomycin, netilmicin, ribostamycin, sisomicin, spectinomycin, streptomycin, and tobramycin.

Lincosamides

Lincosamides include, but are not limited to, clindamycin and lincomycin.

Streptogramins

Streptogramins include, but are not limited to quinupristin.

Glycopeptides

Glycopeptides include, but are not limited to vancomycin.

Polymyxins

Polymyxins include but are not limited to colisitin.

More examples include fosfomycin, penicillins, cephalosporins, carbapenems, penems, and carbacephems.

In some embodiments, the antibiotic may be administered following aerosol formation and inhalation. Thus, this method of treatment is especially appropriate for the treatment of obstructive pulmonary disorders that include pulmonary infections where such infections include microbial strains that are difficult to treat using an agent delivered parenterally due to the need for high parenteral dose levels (which can cause undesirable side effects), or due to lack of any clinically effective antimicrobial agents. In one such embodiment, this method may be used to administer an antibiotic directly to the site of an infection. Such a method may reduce systemic exposure and maximizes the amount of antibiotic to the site of infection. This method is also appropriate for treating infections involving microbes that are susceptible to antimicrobials as a way of reducing the frequency of selection of resistant microbes. This method is also appropriate for treating infections involving microbes that are otherwise resistant to antibiotic agents as a way of increasing the amount of antimicrobial at the site of microbial infection. A subject may be identified as infected with bacteria that are capable of developing resistance by diagnosing the subject as having symptoms that are characteristic of a bacterial infection with a bacteria species known to have resistant strains or a with a bacteria that is a member of group that are known to have resistant strains. Alternatively, the bacteria may be cultured and identified as a species known to have resistant strains or a bacteria that is a member of group that are known to have resistant strains.

In some embodiments, the aerosol antibiotic is administered at a level sufficient to overcome the emergence resistance in bacteria or increase killing efficiency such that resistance does not have the opportunity to develop.

In some embodiments, the antibiotic therapy may be administered as a treatment or prophylaxis in combination or alternating therapeutic sequence with other aerosol, oral or parenteral antibiotics. By non-limiting example this may include any of the antibiotic classes and agents described above.

In addition, compositions and methods provided herein can include the aerosol antibiotic therapy administered as a treatment or prophylaxis in combination or alternating therapeutic sequence with an additional active agent. As discussed above, some such additional agents can include antibiotics. More additional agents can include bronchodilators, anticholinergics, glucocorticoids, eicosanoid inhibitors, and combinations thereof. Examples of bronchodilators include salbutamol, levosalbuterol, terbutaline, fenoterol, terbutlaine, pirbuterol, procaterol, bitolterol, rimiterol, carbuterol, tulobuterol, reproterol, salmeterol, formoterol, arformoterol, bambuterol, clenbuterol, indacterol, theophylline, roflumilast, cilomilast. Examples of anticholinergics include ipratropium, and tiotropium. Examples of glucocorticoids include prednisone, fluticasone, budesonide, mometasone, ciclesonide, and beclomethasone. Examples of eicosanoids include montelukast, pranlukast, zafirlukast, zileuton, ramatroban, and seratrodast. More additional agents can include pulmozyme, hypertonic saline, agents that restore chloride channel function in CF, inhaled beta-agonists, inhaled antimuscarinic agents, inhaled corticosteroids, and inhaled phosphodiesterase inhibitors. Another example of an additional agent includes seretide. In some embodiments, the aerosol antibiotic therapy administered as a treatment or prophylaxis may be used in combination or alternating therapeutic sequence with an additional active agent. In more embodiments, the additional active agent may be administered as a treatment, alone, co-formulated, or administered with the aerosol antibiotic therapy.

Some embodiments of include methods for reducing the frequency of acute exacerbations of a pulmonary disorder selected from chronic obstructive pulmonary disease (COPD) and chronic bronchitis (CB) in a population of humans having said disorder. Some such embodiments include administering to the population an aerosolized antibiotic, wherein the frequency of acute exacerbations for the population is less than the frequency of acute exacerbations for a population having from an acute exacerbation of COPD or CB that is not treated with the aerosol. In some embodiments, the acute exacerbation comprises an increase in an EXACT PRO score. For example, the acute exacerbation can include an increase of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, points above baseline of an EXACT PRO score for at least 1 day, for at least 2 consecutive days, and for at least 3 consecutive days.

In some embodiments, the frequency of the acute exacerbation is reduced by at least about 50%, at least about 40%, at least about 30%, at least about 20%, at least about 10%. In some embodiments, the frequency of the acute exacerbation is reduced by at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2%, and at least about 1%.

Some embodiments include methods for increasing the period of time between acute exacerbations of a pulmonary disorder selected from chronic obstructive pulmonary disease (COPD) and chronic bronchitis (CB) in a population of humans having said disorder. In some embodiments, the method include administering to said population an aerosolized antibiotic. In some embodiments, the period of time between acute exacerbations for the population is greater than the period of time between acute exacerbations for a population having from an acute exacerbation of COPD or CB that is not treated with the aerosol.

In some embodiments, the acute exacerbation comprises an increase in an EXACT PRO score. For example, the acute exacerbation can include an increase of at least about 1, at least about 2, at least about 3, at least about 4, at least about 5, at least about 6, at least about 7, at least about 8, at least about 9, at least about 10, at least about 11, at least about 12, at least about 13, at least about 14, and at least about 15, points above baseline of an EXACT PRO score for at least 1 day, for at least 2 consecutive days, and for at least 3 consecutive days.

In some embodiments an increase in the period between acute exacerbations is at least about 200 days, at least about 150 days, at least about 140 days, at least about 130 days, at least about 120 days, at least about 110 days, at least about 10 days, at least about 90 days, at least about 80 days, at least about 70 days, at least about 60 days, at least about 50 days, at least about 40 days, at least about 30 days, at least about 20 days, at least about 10 days. In some embodiments, the increase in the period between acute exacerbations is at least about 10 days, at least about 9 days, at least about 8 days, at least about 7 days, at least about 6 days, at least about 5 days, at least about 4 days, at least about 3 days, at least about 2 days, and at least about 1 day.

Some embodiments include methods for reducing the frequency of hospitalizations associated with an acute exacerbation of a pulmonary disorder selected from chronic obstructive pulmonary disease (COPD) and chronic bronchitis (CB) in a population of humans having said disorder. Some such methods include administering to said population an aerosolized antibiotic, wherein the frequency of hospitalizations associated with an acute exacerbation for the population is less than the frequency of hospitalizations associated with an acute exacerbation for a population having from an acute exacerbation of COPD that is not treated with the aerosol.

In some such embodiments, the frequency of hospitalizations is reduced by at least about 50%, at least about 40%, at least about 30%, at least about 20%, and at least about 10%. In some embodiments, the frequency of hospitalizations is reduced by at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2%, and at least about 1%.

Some embodiments include the use of an aerosolized antibiotic for reducing the frequency of acute exacerbations of a pulmonary disorder selected from chronic obstructive pulmonary disease (COPD) and chronic bronchitis (CB) in a population of humans having said disorder, wherein the frequency of acute exacerbations for the population is less than the frequency of acute exacerbations for a population having from an acute exacerbation of COPD or CB that is not treated with the aerosol.

Some embodiments include the use of an aerosolized antibiotic for increasing the period of time between acute exacerbations of a pulmonary disorder selected from chronic obstructive pulmonary disease (COPD) and chronic bronchitis (CB) in a population of humans having said disorder, wherein the period of time between acute exacerbations for the population is greater than the period of time between acute exacerbations for a population having from an acute exacerbation of COPD or CB that is not treated with the aerosol.

Some embodiments include the use of an aerosolized antibiotic for reducing the frequency of hospitalizations associated with an acute exacerbation of a pulmonary disorder selected from chronic obstructive pulmonary disease (COPD) and chronic bronchitis (CB) in a population of humans having said disorder, wherein the frequency of hospitalizations associated with an acute exacerbation for the population is less than the frequency of hospitalizations associated with an acute exacerbation for a population having from an acute exacerbation of COPD that is not treated with the aerosol.

Some embodiments include methods for reducing the rate of acute exacerbations of a pulmonary disorder selected from chronic obstructive pulmonary disease (COPD) and chronic bronchitis (CB) in a population of humans having said disorder. Some such embodiments include administering to the population an aerosolized antibiotic. In some such embodiments, the rate of exacerbations in the population is less than about 12 exacerbations/patient year, less than about 11 exacerbations/patient year, less than about 10 exacerbations/patient year, less than about 9 exacerbations/patient year, less than about 8 exacerbations/patient year, less than about 7 exacerbations/patient year, less than about 6 exacerbations/patient year, less than about 5 exacerbations/patient year, less than about 4 exacerbations/patient year, less than about 3 exacerbations/patient year, less than about 2 exacerbations/patient year, and less than about 1 exacerbations/patient year. In some such embodiments, the rate of exacerbations in the population is less than about 3.0 exacerbations/patient year, less than about 2.9 exacerbations/patient year, less than about 2.8 exacerbations/patient year, less than about 2.7 exacerbations/patient year, less than about 2.6 exacerbations/patient year, less than about 2.5 exacerbations/patient year, less than about 2.4 exacerbations/patient year, less than about 2.3 exacerbations/patient year, less than about 2.2 exacerbations/patient year, less than about 2.1 exacerbations/patient year, less than about 2.0 exacerbations/patient year, less than about 1.9 exacerbations/patient year, less than about 1.8 exacerbations/patient year, less than about 1.7 exacerbations/patient year, less than about 1.6 exacerbations/patient year, less than about 1.5 exacerbations/patient year, less than about 1.4 exacerbations/patient year, less than about 1.3 exacerbations/patient year, less than about 1.2 exacerbations/patient year, less than about 1.1 exacerbations/patient year, or, less than about 1.0 exacerbations/patient year. In some such embodiments, the rate of exacerbations in the population is less than about 1.0 exacerbations/patient year, less than about 0.9 exacerbations/patient year, less than about 0.8 exacerbations/patient year, less than about 0.7 exacerbations/patient year, less than about 0.6 exacerbations/patient year, less than about 0.5 exacerbations/patient year, less than about 0.4 exacerbations/patient year, less than about 0.3 exacerbations/patient year, less than about 0.2 exacerbations/patient year, and less than about 0.1 exacerbations/patient year,

In the context of a population of humans, a “patient year” refers to the number humans in the population over the course of a year. Thus, for example, the number of exacerbations/patient year refers to the total number of exacerbations in the population during one year divided by the number patients in the population. In other words, 2 exacerbations/patient year would indicate that, on average, each patient in the population experiences 2 exacerbations per year.

Some embodiments include methods for increasing the period of time between acute exacerbations of a pulmonary disorder selected from chronic obstructive pulmonary disease (COPD) and chronic bronchitis (CB) in a population of humans having the disorder. Some such methods include administering to the population an aerosolized antibiotic. In some embodiments the median period of time between acute exacerbations for the population is greater than about 300 days, greater than about 250 days, greater than about 200 days, greater than about 150 days, greater than about 100 days, or greater than about 50 days. In some embodiments the median period of time between acute exacerbations for the population is greater than about 287 days, or greater than about 281 days.

In some of the foregoing embodiments, an acute exacerbation comprises an increase in an EXACT PRO score. In some embodiments, an acute exacerbation comprises an increase of at least 2 standard deviations above an individual patient's average baseline score for at least 2 consecutive days, with the first of the two days serving as the day of onset.

In some embodiments, an acute exacerbation comprises an increase in an EXACT PRO score of about 12 points above baseline for two consecutive days. In some embodiments, an acute exacerbation comprises an increase above baseline in an EXACT PRO score of about 12 points/day, or an increase above baseline in an EXACT PRO score of about 6 points/day. In some embodiments, an acute exacerbation comprises an increase above baseline in an EXACT PRO score of about 12 points in 1 day. In some embodiments, an acute exacerbation comprises an increase above baseline in an EXACT PRO score of about 6 points in 1 day. In some embodiments, an acute exacerbation comprises an increase above baseline in an average EXACT PRO score of about 6 points/day over 3 consecutive days, or an increase in an average EXACT PRO score of about 12 points/day over 3 consecutive days.

In some embodiments, an acute exacerbation comprises an increase above baseline in an EXACT PRO score for 1 day of at least about 2 points, at least about 3 points, at least about 4 points, at least about 5 points, at least about 6 points, at least about 7 points, at least about 8 points, at least about 9 points, at least about 10 points, at least about 11 points, and at least about 12 points. In some embodiments, an acute exacerbation comprises an increase above baseline in an EXACT PRO score for two consecutive days of at least about 2 points, at least about 3 points, at least about 4 points, at least about 5 points, at least about 6 points, at least about 7 points, at least about 8 points, at least about 9 points, at least about 10 points, at least about 11 points, and at least about 12 points. In some embodiments, an acute exacerbation comprises an increase above baseline in an EXACT PRO score for three consecutive days of at least about 2 points, at least about 3 points, at least about 4 points, at least about 5 points, at least about 6 points, at least about 7 points, at least about 8 points, at least about 9 points, at least about 10 points, at least about 11 points, and at least about 12 points. In some embodiments, an acute exacerbation comprises an increase above baseline in an EXACT PRO score for four consecutive days of at least about 2 points, at least about 3 points, at least about 4 points, at least about 5 points, at least about 6 points, at least about 7 points, at least about 8 points, at least about 9 points, at least about 10 points, at least about 11 points, and at least about 12 points. In some embodiments, an acute exacerbation comprises an increase above baseline in an EXACT PRO score for five consecutive days of at least about 2 points, at least about 3 points, at least about 4 points, at least about 5 points, at least about 6 points, at least about 7 points, at least about 8 points, at least about 9 points, at least about 10 points, at least about 11 points, and at least about 12 points.

Some embodiments include methods for reducing the rate of respiratory-related hospitalizations in a population of humans having a disorder selected from chronic obstructive pulmonary disease (COPD) and chronic bronchitis (CB). Some such methods include administering to the population an aerosolized antibiotic. In some embodiments, the rate of respiratory-related hospitalizations in the population is less than about 0.80 respiratory-related hospitalizations/patient year, less than about 0.75 respiratory-related hospitalizations/patient year, less than about 0.70 respiratory-related hospitalizations/patient year, less than about 0.65 respiratory-related hospitalizations/patient year, less than about 0.60 respiratory-related hospitalizations/patient year, less than about 0.55 respiratory-related hospitalizations/patient year, less than about 0.55 respiratory-related hospitalizations/patient year, less than about 0.45 respiratory-related hospitalizations/patient year, less than about 0.40 respiratory-related hospitalizations/patient year, less than about 0.35 respiratory-related hospitalizations/patient year, less than about 0.30 respiratory-related hospitalizations/patient year, less than about 0.25 respiratory-related hospitalizations/patient year, less than about 0.20 respiratory-related hospitalizations/patient year, less than about 0.15 respiratory-related hospitalizations/patient year, or less than about 0.10 respiratory-related hospitalizations/patient year.

In some embodiments, the rate of respiratory-related hospitalizations comprises an exacerbation-related hospitalization rate. In some embodiments, the exacerbation-related hospitalization rate is less than about 0.5 exacerbation-related hospitalization/patient year, less than about 0.4 exacerbation-related hospitalization/patient year, less than about 0.3 exacerbation-related hospitalization/patient year, less than about 0.2 exacerbation-related hospitalization/patient year, or less than about 0.1 exacerbation-related hospitalization/patient year. In some embodiments, the exacerbation-related hospitalization rate is less than about 0.42 exacerbation-related hospitalization/patient year, or less than about 0.27 exacerbation-related hospitalization/patient year.

In some embodiments of the methods, compositions, and uses provided herein, the aerosolized antibiotic and/or the antibiotic therapy comprises an aerosol of a solution comprising levofloxacin or ofloxacin and a divalent or trivalent cation.

In some embodiments of the methods, compositions, and uses provided herein, a population, e.g., a population of humans, can include patients that have recently experienced an acute exacerbation of a pulmonary disorder. For example, a population can include patients that have experienced an acute exacerbation of a pulmonary disorder within at least about 1 day, 5 days, 10 days, 15, days, 20 days, 25 days, and 30 days prior to receiving aerosolized antibiotic therapy or other therapy provided herein. In some embodiments, a population can include patients that have experienced an acute exacerbation of a pulmonary disorder within at least about 1 week, 2 weeks, 3 weeks, 4 weeks, 5 weeks, and 6 weeks prior to receiving antibiotic therapy or other therapy provided herein. In some embodiments, a population, can include patients that have experienced an acute exacerbation of a pulmonary disorder within at least about 1 month, 2 months, 3 months, 4 months, 5 months, 6 months, 7 months, 8 months, 9 months, 10 months, 11 months and 12 months prior to receiving antibiotic therapy or other therapy provided herein.

In some embodiments of the methods, compositions, and uses provided herein, a population, e.g., a population of humans, can include patients that have recently received treatment for an acute exacerbation of a pulmonary disorder. For example, a population can include patients that have received treatment for an acute exacerbation of a pulmonary disorder within at least about 1 day, 5 days, 10 days, 15, days, 20 days, 25 days, and 30 days prior to receiving antibiotic therapy or other therapy provided herein. In some embodiments, a population can include patients that have received treatment for an acute exacerbation of a pulmonary disorder within at least about 1 week, 2 weeks, 3 weeks, 4 weeks, 5 weeks, and 6 weeks prior to receiving antibiotic therapy or other therapy provided herein. In some embodiments, a population can include patients that have received treatment for an acute exacerbation of a pulmonary disorder within at least about 1 month, 2 months, 3 months, 4 months, 5 months, 6 months, 7 months, 8 months, 9 months, 10 months, 11 months and 12 months prior to receiving antibiotic therapy or other therapy provided herein.

In some embodiments of the methods, compositions, and uses provided herein, the duration of a therapy, e.g., an aerosolized antibiotic therapy can include at least about 1 day/month, at least about 2 days/month, at least about 3 days/month, at least about 4 days/month, at least about 5 days/month, at least about 6 days/month, at least about 7 days/month, at least about 8 days/month, at least about 9 days/month, at least about 10 days/month, at least about 11 days/month, at least about 12 days/month, at least about 13 days/month, at least about 14 days/month, at least about 15 days/month, at least about 16 days/month, at least about 17 days/month, at least about 18 days/month, at least about 19 days/month, at least about 20 days/month, at least about 21 days/month, at least about 22 days/month, at least about 23 days/month, at least about 24 days/month, at least about 25 days/month, at least about 26 days/month, at least about 27 days/month, at least about 28 days/month, at least about 29 days/month, at least about 30 days/month, and at least about 31 days/month.

### Pharmaceutical Compositions

For purposes of the method described herein, a fluoroquinolone agent formulated with a divalent or trivalent cation having improved pulmonary bioavailability may be administered using an inhaler. In some embodiments, a fluoroquinolone disclosed herein is produced as a pharmaceutical composition suitable for aerosol formation, good taste, storage stability, and patient safety and tolerability. In some embodiments, the isoform content of the manufactured fluoroquinolone may be optimized for tolerability, antimicrobial activity and stability.

Formulations can include a divalent or trivalent cation. The divalent or trivalent cation can include, for example, magnesium, calcium, zinc, copper, aluminum, and iron. In some embodiments, the solution comprises magnesium chloride, magnesium sulfate, zinc chloride, or copper chloride. In some embodiments, the divalent or trivalent cation concentration can be from about 25 mM to about 400 mM, from about 50 mM to about 400 mM, from about 100 mM to about 300 mM, from about 100 mM to about 250 mM, from about 125 mM to about 250 mM, from about 150 mM to about 250 mM, from about 175 mM to about 225 mM, from about 180 mM to about 220 mM, and from about 190 mM to about 210 mM. In some embodiments, the magnesium chloride, magnesium sulfate, zinc chloride, or copper chloride can have a concentration from about 5% to about 25%, from about 10% to about 20%, and from about 15% to about 20%. In some embodiments, the ratio of fluoroquinolone to divalent or trivalent cation can be 1:1 to 2:1 or 1:1 to 1:2.

Non-limiting fluoroquinolones for use as described herein include levofloxacin, ofloxacin, ciprofloxacin, enoxacin, gatifloxacin, gemifloxacin, lomefloxacin, moxifloxacin, norfloxacin, pefloxacin, sparfloxacin, garenoxacin, sitafloxacin, and DX-619.

The formulation can have a fluoroquinolone concentration, for example, levofloxacin or ofloxacin, greater than about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, and about 200 mg/ml. In some embodiments, the formulation can have a fluoroquinolone concentration, for example, levofloxacin or ofloxacin, from about 50 mg/ml to about 200 mg/ml, from about 75 mg/ml to about 150 mg/ml, from about 80 mg/ml to about 125 mg/ml, from about 80 mg/ml to about 120 mg/ml, from about 90 mg/ml to about 125 mg/ml, from about 90 mg/ml to about 120 mg/ml, and from about 90 mg/ml to about 110 mg/ml.

The formulation can have an osmolality from about 300 mOsmol/kg to about 500 mOsmol/kg, from about 325 mOsmol/kg to about 450 mOsmol/kg, from about 350 mOsmol/kg to about 425 mOsmol/kg, and from about 350 mOsmol/kg to about 400 mOsmol/kg. In some embodiments, the osmolality of the formulation is greater than about 300 mOsmol/kg, about 325 mOsmol/kg, about 350 mOsmol/kg, about 375 mOsmol/kg, about 400 mOsmol/kg, about 425 mOsmol/kg, about 450 mOsmol/kg, about 475 mOsmol/kg, and about 500 mOsmol/kg.

The formulation can have a pH from about 4.5 to about 8.5, from about 5.0 to about 8.0, from about 5.0 to about 7.0, from about 5.0 to about 6.5, from about 5.5 to about 6.5, and from 6.0 to about 6.5.

The formulation can comprise a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like), or auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). In some embodiments, the formulation can lack a conventional pharmaceutical carrier, excipient or the like. Some embodiments include a formulation lacking lactose. Some embodiments comprise lactose at a concentration less than about 10%, 5%, 1%, or 0.1%. In some embodiments, the formulation can consist essentially of levofloxacin or ofloxacin and a divalent or trivalent cation.

In some embodiments, a formulation can comprise a levofloxacin concentration between about 75 mg/ml to about 150 mg/ml, a magnesium chloride concentration between about 150 mM to about 250 mM, a pH between about 5 to about 7; an osmolality of between about 300 mOsmol/kg to about 500 mOsmol/kg, and lacks lactose.

In some embodiments, a formulation comprises a levofloxacin concentration about 100 mg/ml, a magnesium chloride concentration about 200 mM, a pH about 6.2, and an osmolality of about 383 mOsmol/kg. In some embodiments, a formulation consists essentially of a levofloxacin concentration about 100 mg/ml, a magnesium chloride concentration about 200 mM, a pH about 6.2, and an osmolality of about 383 mOsmol/kg. In some embodiments, a formulation consists of a levofloxacin concentration about 100 mg/ml, a magnesium chloride concentration about 200 mM, a pH about 6.2, and an osmolality of about 383 mOsmol/kg.

### Administration

The aerosolized antimicrobials may be administered at a therapeutically effective dosage, e.g., a dosage sufficient to provide the treatment outcomes described herein. The amount of active compound administered may be dependent on the subject being treated, the severity of the affliction, the manner and schedule of administration and the judgment of the prescribing physician; for example, a likely dose range for aerosol administration of levofloxacin would be about 20 to 300 mg BID (twice daily), the active agents being selected for longer or shorter pulmonary half-lives, respectively.

Administration of the antimicrobial agents disclosed herein or the pharmaceutically acceptable salts thereof can be via aerosol inhalation. Methods, devices and compositions for delivery are described in U.S. Patent Application Publication No. 2006-0276483, incorporated by reference in its entirety.

Pharmaceutically acceptable compositions include solid, semi-solid, liquid and aerosol dosage forms, such as, for example, powders, liquids, suspensions, complexations, liposomes, particulates, or the like. Preferably, the compositions are provided in unit dosage forms suitable for single administration of a precise dose.

The aerosolized antimicrobial agent can be administered either alone or in some alternatives, in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, the pharmaceutical formulation will contain about 0.005% to 95%, preferably about 0.5% to 50% by weight of a compound of the invention. Actual methods of preparing such dosage forms are known, or will be apparent, to those skilled in this art; for example, see Remington's Pharmaceutical Sciences, Mack Publishing Company, Easton, Pa.

In one preferred embodiment, the compositions will take the form of a unit dosage form such as vial containing a liquid, solid to be suspended, dry powder, lyophilate, or other composition.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution or suspension. Solutions to be aerosolized can be prepared in conventional forms, either as liquid solutions or suspensions, as emulsions, or in solid forms suitable for dissolution or suspension in liquid prior to aerosol production and inhalation. The percentage of active compound contained in such aerosol compositions is highly dependent on the specific nature thereof, as well as the activity of the compound and the needs of the subject. However, percentages of active ingredient of 0.01% to 90% in solution are employable, and will be higher if the composition is a solid, which will be subsequently diluted to the above percentages. In some embodiments, the composition will comprise 1.0%-50.0% of the active agent in solution.

Compositions described herein can be administered with a frequency of about 1, 2, 3, 4, or more times daily, 1, 2, 3, 4, 5, 6, 7 or more times weekly, 1, 2, 3, 4, 5, 6, 7, 8, 9, 10 or more times monthly. In particular embodiments, the compositions are administered twice daily.

### Aerosol Delivery

For pulmonary administration, the upper airways are avoided in favor of the middle and lower airways. Pulmonary drug delivery may be accomplished by inhalation of an aerosol through the mouth and throat. Particles having a mass median aerodynamic diameter (MMAD) of greater than about 5 microns generally do not reach the lung; instead, they tend to impact the back of the throat and are swallowed and possibly orally absorbed. Particles having diameters of about 2 to about 5 microns are small enough to reach the upper-to mid-pulmonary region (conducting airways), but are too large to reach the alveoli. Smaller particles, i.e., about 0.5 to about 2 microns, are capable of reaching the alveolar region. Particles having diameters smaller than about 0.5 microns can also be deposited in the alveolar region by sedimentation, although very small particles may be exhaled.

In one embodiment, a nebulizer is selected on the basis of allowing the formation of an aerosol of a fluoroquinolone antimicrobial agent disclosed herein having an MMAD predominantly between about 2 to about 5 microns. In one embodiment, the delivered amount of fluoroquinolone antimicrobial agent provides a therapeutic effect for respiratory infections. The nebulizer can deliver an aerosol comprising a mass median aerodynamic diameter from about 2 microns to about 5 microns with a geometric standard deviation less than or equal to about 2.5 microns, a mass median aerodynamic diameter from about 2.5 microns to about 4.5 microns with a geometric standard deviation less than or equal to about 1.8 microns, and a mass median aerodynamic diameter from about 2.8 microns to about 4.3 microns with a geometric standard deviation less than or equal to about 2 microns. In some embodiments, the aerosol can be produced using a vibrating mesh nebulizer. An example of a vibrating mesh nebulizer includes the PARI E-FLOW® nebulizer. More examples of nebulizers are provided in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303 all of which are hereby incorporated by reference in their entireties. More commercial examples of nebulizers that can be used with the formulations described herein include Respirgard II®, Aeroneb®, Aeroneb® Pro, and Aeroneb® Go produced by Aerogen; AERx® and AERx Essence™ produced by Aradigm; Porta-Neb®, Freeway Freedom™, Sidestream, Ventstream and I-neb produced by Respironics, Inc.; and PARI LC-Plus®, PART LC-Star®, produced by PARI, GmbH. By further non-limiting example, U.S. Pat. No. 6,196,219, is hereby incorporated by reference in its entirety.

The amount of antibiotic that can be administered to the lungs can include at least about 20 mg, about 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, and about 800 mg.

The aerosol can be administered to the lungs in less than about 10 minutes, about 5 minutes, about 4 minutes, about 3 minutes, about 2 minutes, and about 1 minute.

### Indications

Methods and compositions described herein can be used to treat obstructive pulmonary disorders, for example, chronic obstructive pulmonary disease, chronic bronchitis (CB), and some asthmas.

In some embodiments, the incidence and/or severity of acute exacerbations can be reduced by the methods provided herein. Acute exacerbations can be indicated by the presence of a symptom that includes increased sputum production, more purulent sputum, change in sputum color, increased coughing, increased wheezing, chest tightness, reduced exercise tolerance, increased fatigue, fluid retention, acute confusion, worsened dyspnea, and combinations thereof. In addition, an acute exacerbation can include a symptomatic respiratory deterioration requiring treatment with antibiotic agents, systemic corticosteroids, hospitalization, or a combination of these treatments.

In some embodiments, the incidence of acute exacerbations in a subject is reduced. The period between acute exacerbations can be increased by about 1 day, 2 days, 3 days, 4 days, 5 days, 6 days, 7 days, 8 days, 9 days, and 10 days, will be increased about 1 week, 2 weeks, 3 weeks, 4 weeks, 5 weeks, 6 weeks, 7 weeks, 8 weeks, 9 weeks, and 10 weeks, will be increased by about 1 month, 2 months, 3 months, 4 months, 5 months, 6 months, 7 months, 8 months, 9 months, 10 months, 11 months, and 12 months, will be increased by 1 year, and 2 years. In some embodiments, acute exacerbations in a patient will be prevented.

In some embodiments, the methods and compositions provided herein can reduce the average hospitalization time for a patient suffering from an acute exacerbation. The average hospitalization time can be measured, for example, as the time from the onset of treatment in the hospital to the time the acute exacerbation is sufficiently lessened or eliminated such that the patient is discharged from the hospital. In such embodiments, the average hospitalization time for the patient is less than the average hospitalization time for a patient suffering from an acute exacerbation that is not treated with the antibiotic. The average hospitalization time for a patient suffering from an acute exacerbation can be reduced by at least about 10%, 20%, 30%, 40%, 50%, 60%, 70%, 80%, and 90%. The average hospitalization time for a patient suffering from an acute exacerbation can be reduced by at least about 1 day, 2 days, 3, days, 4 days, 5 days, 6 days, 7 days, 8 days, 9 days, and 10 days. The average hospitalization time for a patient suffering from an acute exacerbation can be less than about 1 day, 5 days, and 10 days.

The severity and/or duration of acute exacerbations can be measured by a variety of methods. An example is an Exacerbations of Chronic Obstructive Pulmonary Disease Tool (EXACT) with a 14-item patient-reported outcome (PRO) measure designed to standardize the measurement approach for evaluating the frequency, severity, and duration of acute exacerbations of COPD in clinical trials (Jones, P. et al., (2007) “Quantifying of severity of exacerbations in chronic obstructive pulmonary disease: adaptations to the definition to allow quantification” Proc Am Thorac Soc. 4:597-601, incorporated by reference in its entirety). The EXACT PRO can include a daily questionnaire completed by patients to report any symptom and exacerbation. Patients can use standard definitions and items in the questionnaire that relate to breathlessness, cough and sputum, chest symptoms, global symptoms, healthcare intervention and changes in medications over the day. In such methods, an exacerbation can include an increase of at least 2 standard deviations above an individual patient's average baseline score for at least 2 consecutive days, with the first of the two days serving as the day of onset. PROs can assess any aspect of a patient's health status that comes directly from the patient (i.e., without the interpretation of the patient's responses by a physician or anyone else) and can be used to measure the impact of an intervention on one or more aspects of patient's health status. In COPD or CB, PROs can quantify symptoms recorded during an exacerbation, whether or not the patient seeks medical attention. An increase in the EXACT PRO score can be associated with an exacerbation and is characterized by worsening of chronic symptoms including, but not limited to, breathlessness, cough, chest tightness, and nighttime waking. In more examples, the EXACT PRO can include terms and scores that relate to the presence of a symptom that includes increased sputum production, more purulent sputum, change in sputum color, increased coughing, increased wheezing, chest tightness, reduced exercise tolerance, increased fatigue, fluid retention, acute confusion, worsened dyspnea, and combinations thereof. In addition, the EXACT can include terms and scores that relate to a symptomatic respiratory deterioration requiring treatment with antibiotic agents, systemic corticosteroids, hospitalization, or a combination of these treatments.

More examples of methods to measure the severity and/or duration of acute exacerbations include the use of indices such as the St. George's Respiratory Questionnaire (SGRQ), sign and symptoms questionnaire (Meguro et al., (2007) “Development and validation of an improved, COPD-specific version of the St George's Respiratory Questionnaire” Chest 132:456-63, incorporated by reference in its entirety). The SGRQ is a disease-specific instrument designed to measure impact on overall health, daily life, and perceived well-being and has been developed for use by patients with fixed and reversible airway obstruction. Scores for these components and the summary score based on a 100-point scale. In some embodiments, methods and compositions provided herein include achieving a more advantageous SQRQ index in patients with an obstructive pulmonary disorder.

In more embodiments, the methods and compositions provided herein include achieving a more advantageous BODE index in patients with an obstructive pulmonary disorder. The BODE index is a multidimensional grading system that assesses the respiratory, perceptive, and systemic aspects of COPD that would better categorize the illness. The index relates to a body-mass index (B), the degree of airflow obstruction (O), functional dyspnea (D), and exercise capacity (E) (Celli B R, et al., The body-mass index, airflow obstruction, dyspnea, and exercise capacity index in chronic obstructive pulmonary disease. N Engl J Med. 2004 Mar. 4; 350 (10):1005-12, incorporated by reference in its entirety).

In even more embodiments, the methods and compositions provided herein include achieving a more advantageous index in patients with an obstructive pulmonary disorder, wherein the index includes the Modified Medical Research Council (MMRC) scale, baseline dyspnea index (BDI) and the oxygen cost diagram (OCD) (Chhabra et al. (2009) “Evaluation of three scales of dyspnea in chronic obstructive pulmonary disease” Ann Thorac Med 4:128-32, incorporated by reference in its entirety).

More embodiments include treating an infection comprising one or more bacteria that can include Pseudomonas aeruginosa, Pseudomonas fluorescens, Pseudomonas acidovorans, Pseudomonas alcaligenes, Pseudomonas putida, Stenotrophomonas maltophilia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Helicobacter pylori, Campylobacter fetus, Campylobacter jejuni, Campylobacter coli, Borrelia burgdorferi, Vibrio cholera, Vibrio parahaemolyticus, Legionella pneumophila, Listeria monocytogenes, Neisseria gonorrhoeae, Neisseria meningitidis, Burkholderia cepacia, Francisella tularensis, Kingella, and Moraxella. In some embodiments, the pulmonary infection can include a gram-negative anaerobic bacteria. In more embodiments, the pulmonary infection can include one or more of the bacteria selected from the group consisting of Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, and Bacteroides splanchnicus. In some embodiments, the pulmonary infection can include a gram-positive bacteria. In some embodiments, the pulmonary infection can include one or more of the bacteria selected from the group consisting of Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus. In some embodiments, the pulmonary infection can include a gram-positive anaerobic bacteria. In some embodiments, the pulmonary infection can include one or more bacteria selected from the group consisting of Clostridium difficile, Clostridium perfringens, Clostridium tetini, and Clostridium botulinum. In some embodiments, the pulmonary infection can include an acid-fast bacteria. In some embodiments, the pulmonary infection can include one or more bacteria selected from the group consisting of Mycobacterium tuberculosis, Mycobacterium avium, Mycobacterium intracellulare, and Mycobacterium leprae. In some embodiments, the pulmonary infection can include an atypical bacteria. In some embodiments, the pulmonary infection can include one or more bacteria selected from the group consisting of Chlamydia pneumoniae and Mycoplasma pneumoniae.

### EXAMPLES

### Example 1

### Phase 1b Clinical Study on Administration of Levofloxacin with MgCl₂ to CB Patients

A Phase 1b, multi-center, randomized, single-blind, placebo-controlled study was carried out to evaluate two dosage regimens of levofloxacin formulated with MgCl₂ (240 mg QD, and 240 mg BID) administered for 5 days by aerosol route stable CB patients. The formulations used for the study drugs are shown in Table 1.

TABLE 1

| | Levofloxacin with MgCl₂ | |
|---|---|---|
| | 100 mg/ml | Placebo |
| Levofloxacin, mg/ml (mM) | 100 (272) | 0 |
| Magnesium, mg/ml (mM) | 4.9 (200) | 0 |
| Chloride, mg/ml (mM) | 14.2 (400) | 0 |
| pH | 6-8 | 6-8 |
| Osmolality, mOsm/kg | 300-500 | 300-500 |
| Saline | n/a | 0.9% |

Study drugs were administered by aerosol using a modified PARI eFlow® device (manufactured by PARI GmbH Moosstrasse 3, D-82319 Starnberg, Germany). The PART eFlow® nebulizer uses a vibrating mesh to nebulize the medication and deliver medication with a particle size of approximately 3.5-4.0 μm. Patients were included in the study if they had symptoms consistent with CB, but had no documented episode of acute exacerbation of CB requiring treatment within 60 days before Day 1. Patients were excluded from the trial if their current use of oral corticosteroids exceeded doses equivalent to 10 mg prednisone/day or 20 mg prednisone every other day. Table 2 summarizes corticosteroid use of patients six months prior to the study.

TABLE 2

| | Levofloxacin | Levofloxacin | |
|---|---|---|---|
| | formulated | formulated | |
| | with MgCl₂ | with MgCl₂ | |
| Adrenergics/drugs for | 240 mg QD | 240 mg BID | Placebo |
| obstructive airways disease: | N = 9 | N = 8 | N = 4 |
| Budesonide with formoterol | 0 | 1 | 0 |
| fumarate | | | |
| Seretide | 3 | 3 | 2 |
| Glucocorticords: | | | |
| Prednisone | 1 | 2 | 1 |
| Triamcinolone | 0 | 1 | 1 |
| Total number of patients that used | 4 | 5 | 3 |
| corticosteroids 6 months prior to | | | |
| study | | | |

### Pulmonary Function Evaluations

All patients underwent pulmonary function tests to determine parameters that included their FVC (forced vital capacity) and FEV₁ (forced expiratory volume in 1 second) according to the spirometry standards of the American Thoracic Society (Pellegrino R, et al. 2005. Interpretative strategies for lung function tests. Eur Respir J 26: 948-968, incorporated by reference in its entirety). Table 3 summarizes FEV₁ data for changes from pre-dose at Day 1 and Day 5, where baseline values were Day 1 pre-dose.

TABLE 3

| | | Levofloxacin | | Levofloxacin | | | |
|---|---|---|---|---|---|---|---|
| | | with MgCl₂ | | with MgCl₂ | | | |
| | | 240 mg QD | | 240 mg BID | | Placebo | |
| | | (n = 9) | | (n = 8) | | (n = 4) | |
| | | | Percent | | Percent | | Percent |
| | | | Change | | Change | | Change |
| | | FEV₁ | From | FEV₁ | From | FEV₁ | From |
| | Parameter | (L) | Baseline | (L) | Baseline | (L) | Baseline |
| Day 1 pre- | Mean (SD) | 1.45 | N/A | 1.37 | N/A | 1.16 | N/A |
| dose | | (0.71) | | (0.70) | | (0.84) | |
| | Median | 1.28 | N/A | 1.12 | N/A | 0.90 | N/A |
| | Minimum, | 0.56, | N/A | 0.78, | N/A | 0.50, | N/A |
| | Maximum | 2.82 | | 2.68 | | 2.34 | |
| Day 1 pre- | Mean (SD) | 1.42 | −5.13 | 1.32 | −4.55 | 1.16 | 0.82 |
| dose to 30 | | (0.81) | (10.11) | (0.71) | (11.74) | (0.83) | (11.46) |
| minutes | Median | 1.17 | −7.75 | 1.12 | −3.25 | 0.90 | 2.78 |
| post-dose | Minimum, | 0.51, | −22.22, | 0.68, | −24.73, | 0.54, | 14.29, |
| | Maximum | 2.86 | 12.75 | 2.63 | 16.67 | 2.31 | 12.00 |
| Day 1 pre- | Mean (SD) | 1.43 | −4.30 | 1.29 | −9.10 | 1.16 | −3.18 |
| dose to 2 | | (0.78) | (10.92) | (0.81) | (14.58) | (0.89) | (15.26) |
| hours post- | Median | 1.15 | 0.00 | 1.02 | −14.97 | 0.89 | 1.64 |
| dose | Minimum, | 0.43, | −23.21, | 0.61, | −27.38, | 0.47, | −25.40, |
| | Maximum | 2.84 | 8.45 | 3.04 | 13.43 | 2.37 | 9.40 |
| Day 5 pre- | Mean (SD) | 1.55 | N/A | 1.37 | N/A | 1.14 | N/A |
| dose | | (0.90) | | (0.66) | | (0.86) | |
| | Median | 1.25 | N/A | 1.17 | N/A | 0.82 | N/A |
| | Minimum, | 0.58, | N/A | 0.73, | N/A | 0.54, | N/A |
| | Maximum | 2.94 | | 2.68 | | 2.36 | |
| Day 5 pre- | Mean (SD) | 1.50 | −3.83 | 1.39 | −0.84 | 1.13 | −1.53 |
| dose to 30 | | (0.90) | (5.58) | (0.80) | (13.36) | (0.87) | (5.29) |
| minutes | Median | 1.16 | −3.70 | 1.05 | 0.94 | 0.84 | 0.21 |
| post-dose | Minimum, | 0.60, | −10.00, | 0.75, | −22.68, | 0.49, | −9.26, |
| | Maximum | 2.88 | 3.45 | 3.13 | 16.79 | 2.37 | 2.73 |
| Day 5 pre- | Mean (SD) | 1.52 | −2.60 | 1.28 | −8.71 | 1.17 | 0.38 |
| dose to 2 | | (0.93) | (8.20) | (0.72) | (11.37) | (0.95) | (9.53) |
| hours post- | Median | 1.20 | 0.34 | 0.97 | −12.99 | 0.84 | 3.60 |
| dose | Minimum, | 0.61, | −17.82, | 0.63, | −20.83, | 0.47, | −12.96, |
| | Maximum | 2.96 | 5.17 | 2.84 | 7.14 | 2.53 | 7.27 |

At Baseline, mean and median FEV₁ values were slightly greater in the levofloxacin with MgCl₂ treatment groups compared to the placebo group. The differences observed at Baseline were likely due to the small sample size of the placebo group. The changes observed in FEV₁ and the results of other pulmonary function tests (data not shown) were not significant and not clinically relevant.

### Safety Evaluations

All adverse events were mild or moderate in severity and generally self limiting, with the exception of one severe adverse event. The profile of reported adverse events between the 240 mg QD and 240 mg BID levofloxacin with MgCl₂ were similar. Eleven patients (64.7%) treated with levofloxacin with MgCl₂ experienced at least one adverse event: 6/9 patients (66.7%) in the 240 mg QD group and ⅝ patients (62.5%) in the 240 mg BID group. Unexpectedly, no patients in the placebo reported an adverse event or serious adverse event. The most commonly reported adverse event was dysgeusia with 2 patients (22.2%) reporting dysgeusia in the 240 mg QD group and 4 patients (50.0%) in the 240 mg BID group. Other indicators to evaluate safety of the study drugs such as clinical laboratory results, physical examinations, vital sign measurements, pulse oximetry, pulmonary function tests, and ECGs indicated that the two levofloxacin formulated with MgCl₂ dosage regimens were well tolerated. Overall, levofloxacin formulated with MgCl₂ was well tolerated with similar safety profiles being observed in the 2 dosage regimens of levofloxacin formulated with MgCl₂, 240 mg QD and 240 mg BID administered for 5 days. Safety results from this study supported further evaluation of levofloxacin formulated with MgCl₂ (levofloxacin inhalation solution) in a Phase 2 clinical study to assess the safety and efficacy of levofloxacin formulated with MgCl₂ in the prevention of acute exacerbations in COPD patients.

### Example 2

### Phase 2 Clinical Study: Administration of Levofloxacin Formulated with MgCl₂ to COPD Patients

A clinical study to evaluate the safety, tolerability and efficacy of levofloxacin formulated with MgCl₂ was carried out on COPD patients. The study was a Phase 2, multi-center, randomized, double-blind, placebo-controlled study. Patients were administered 240 mg BID levofloxacin formulated with MgCl₂ or placebo. The formulations of the study drug and placebo are shown in Table 4.

TABLE 4

| | Levofloxacin | |
|---|---|---|
| | formulated with MgCl₂ | Placebo |
| Levofloxacin, mg/ml (mM) | 100 (272) | 0 |
| Magnesium, mg/ml (mM) | 4.9 (200) | 0 |
| Chloride, mg/ml (mM) | 14.2 (400) | 0 |
| pH | 6-8 | 4.5-7.5 |
| Osmolality, mOsm/kg | 300-500 | 270-300 |
| Saline | N/A | 0.9% |
| Riboflavin 5′-phophate, μg/ml | 0 | 4 |

Study drug and placebo were administered using a modified PARI eFlow® nebulizer. The study included a series of at least six, but no more than 12, treatment cycles. Each treatment cycle was 28 days. In each treatment cycle, patients were administered either 240 mg BID levofloxacin with MgCl₂ or placebo for 5 consecutive days.

### Patient Population

Approximately 300 patients were studied. Criteria for including patients in this study included those that had: (1) a history of COPD with mucopurulent sputum (yellow, green or brown/tan) production on most days, even when exacerbation-free; (2) a measured FEV₁<70% of predicted FEV₁ (post-bronchodilator administration) and FEV₁/FVC <0.7 (post-bronchodilator) at screening based on predicted values using age, height and sex using Hankinson and N. Hanes criteria; (3) had at least two documented acute exacerbation episodes during the preceding 12 months prior to Day 1 of Cycle 1, acute exacerbation episodes include episodes that require antibiotic agents, systemic corticosteroids, hospitalization or a combination of these treatments; (4) had no acute exacerbation episode that required treatment within 30 days prior to Day 1 of Cycle 1; (5) a stable treatment history for 30 days prior to Day 1 of Cycle 1, if the patient is receiving chronic therapy with inhaled long acting bronchodilators and/or inhaled or systemic steroids; and (6) a lifetime smoking history of at least 10 pack-years. Criteria for excluding patients from this study included those that had any respiratory tract disorder other than COPD that was considered to be clinically relevant, for example, a history of a primary diagnosis of asthma, bronchial carcinoma, pulmonary tuberculosis, cystic fibrosis or diffuse bronchiectasis.

The patient population included an efficacy evaluable (EE) population, a modified intent to treat (MITT) population, and a pharmacokinetic (PK) population. The EE population included all patients enrolled in the study who completed 80% of their treatment cycles without major protocol violations. The EE population included all enrolled patients without major protocol violations and who do did not fail to complete 2 or more consecutive treatment cycles from Baseline to Final Visit. Completion of a treatment cycle included receiving at least 80% of Study Drug (MP-376/placebo) within each cycle. The MITT population included all patients enrolled in the study that received at least one dose of study drug. The PK population included all patients that received a least one dose of Study Drug and had at least one PK sample collected. Tables 5 and 6 summarize demographic and baseline characteristics for the MITT population and the EE population, respectively.

TABLE 5

| | | | MP-376 | |
|---|---|---|---|---|
| | | Placebo | 240 mg BID | Total |
| MITT Population | | (N = 108) | (N = 214) | (N = 322) |
| Age (years) | Mean (SD) | 63.4 (9.06) | 64.0 (9.14) | 63.8 (9.10) |
| | Median | 64.0 | 65.0 | 64.0 |
| | Min, Max | 41, 81 | 43, 83 | 41, 83 |
| Sex | Male | 56 (51.9%) | 113 (52.8%) | 169 (52.5%) |
| | Female | 52 (48.1%) | 101 (47.2%) | 153 (47.5%) |
| Ethnicity | Hispanic or Latino | 5 (4.6%) | 3 (1.4%) | 8 (2.5%) |
| | Not Hispanic or Latino | 103 (95.4%) | 211 (98.6%) | 314 (97.5%) |
| Race | American Indian or | 0 (0.0%) | 0 (0.0%) | 0 (0.0%) |
| | Alaska Native | | | |
| | Asian | 1 (0.9%) | 1 (0.5%) | 2 (0.6%) |
| | Black or African | 9 (8.3%) | 19 (8.9%) | 28 (8.7%) |
| | American | | | |
| | Native Hawaiian or | 0 (0.0%) | 0 (0.0%) | 0 (0.0%) |
| | Other Pacific Islander | | | |
| | White | 97 (89.8%) | 194 (90.7%) | 291 (90.4%) |
| | Other | 1 (0.9%) | 0 (0.0%) | 1 (0.3%) |
| Weight (kg) | Mean (SD) | 82.5 (20.39) | 83.4 (21.25) | 83.1 (20.94) |
| | Median | 79.2 | 82.4 | 81.2 |
| | Min, Max | 46.3, 132.9 | 44.0, 170.1 | 44.0, 170.1 |
| BMI (kg/m²) | Mean (SD) | 29.1 (6.47) | 29.0 (6.45) | 29.0 (6.45) |
| | Median | 28.2 | 28.6 | 28.5 |
| | Min, Max | 19.0, 53.6 | 14.7, 50.8 | 14.7, 53.6 |
| FEV₁ (L) | Mean (SD) | 1.25 (0.56) | 1.22 (0.55) | 1.23 (0.55) |
| | Median | 1.13 | 1.14 | 1.14 |
| | Min, Max | 0.13, 3.10 | 0.31, 3.50 | 0.13, 3.50 |
| FEV₁ percent predicted | Mean (SD) | 44.3 (17.83) | 43.0 (16.30) | 43.5 (16.81) |
| | Median | 41.9 | 42.0 | 42.0 |
| | Min, Max | 4.3, 90.0 | 12.0, 109.0 | 4.3, 109.0 |
| | <30 | 0 (0.0%) | 0 (0.0%) | 0 (0.0%) |
| | 30-<50 | 0 (0.0%) | 0 (0.0%) | 0 (0.0%) |
| | 50-<80 | 0 (0.0%) | 0 (0.0%) | 0 (0.0%) |
| | ≧80 | 0 (0.0%) | 0 (0.0%) | 0 (0.0%) |
| FVC (L) | Mean (SD) | 2.48 (0.87) | 2.39 (0.78) | 2.42 (0.81) |
| | Median | 2.32 | 2.30 | 2.31 |
| | Min, Max | 0.22, 5.36 | 1.00, 6.09 | 0.22, 6.09 |
| FVC percent predicted | Mean (SD) | 66.4 (17.48) | 63.6 (16.59) | 64.5 (16.91) |
| | Median | 64.7 | 62.6 | 63.8 |
| | Min, Max | 5.7, 113.1 | 19.0, 126.6 | 5.7, 126.6 |
| FEV₁/FVC ratio | Mean (SD) | 0.52 (0.15) | 0.52 (0.18) | 0.52 (0.17) |
| | Median | 0.51 | 0.52 | 0.51 |
| | Min, Max | 0.20, 0.92 | 0.18, 2.25 | 0.18, 2.25 |
| Ever received pneumococcal | Yes | 61 (56.5%) | 140 (65.4%) | 201 (62.4%) |
| vaccine? | | | | |
| | No | 23 (21.3%) | 41 (19.2%) | 64 (19.9%) |
| | Unknown | 24 (22.2%) | 33 (15.4%) | 57 (17.7%) |
| Received influenza vaccine for | Yes | 74 (68.5%) | 152 (71.0%) | 226 (70.2%) |
| 2008/2009 season? | | | | |
| | No | 32 (29.6%) | 62 (29.0%) | 94 (29.2%) |
| | Unknown | 2 (1.9%) | 0 (0.0%) | 2 (0.6%) |
| Current smoker? | Yes | 44 (40.7%) | 99 (46.3%) | 143 (44.4%) |
| | No | 64 (59.3%) | 115 (53.7%) | 179 (55.6%) |
| Number of years of regular | Mean (SD) | 36.4 (11.66) | 39.5 (10.80) | 38.4 (11.17) |
| cigarette smoking | | | | |
| | Median | 36.5 | 40.0 | 40.0 |
| | Min, Max | 7, 67 | 10, 65 | 7, 67 |
| Pack year of smoking [1] | Mean (SD) | 53.5 (30.31) | 59.3 (35.79) | 57.4 (34.11) |
| | Median | 46.0 | 50.0 | 50.0 |
| | Min, Max | 2.5, 180.0 | 10.0, 260.1 | 2.5, 260.1 |
| Prescribed home/oxygen therapy | Yes | 35 (32.4%) | 82 (38.3%) | 117 (36.3%) |
| | No | 73 (67.6%) | 132 (61.7%) | 205 (63.7%) |
| Number of documented acute | Mean (SD) | 2.1 (0.75) | 2.1 (0.65) | 2.1 (0.68) |
| exacerbation episodes in past 12 | Median | 2.0 | 2.0 | 2.0 |
| months | Min, Max | 0, 5 | 0, 5 | 0, 5 |
| Number of Patients taking | Inhaled and systemic | 65 (60.2%) | 142 (66.4%) | 207 (64.3%) |
| specific medications at baseline | corticosteroids | | | |
| for COPD | Long-acting beta | 60 (55.6%) | 124 (57.9%) | 184 (57.1%) |
| | agonists | | | |
| | Long-acting anti- | 51 (47.2%) | 90 (42.1%) | 141 (43.8%) |
| | muscarinic agents | | | |

[1] Calculated as average packs per day multiplied by years of smoking.

TABLE 6

| | | | | MP-376 | | | |
|---|---|---|---|---|---|---|---|
| | | Placebo | | 240 mg BID | | Total | |
| EE Population | | (N = 86) | | (N = 175) | | (N = 261) | |
| Age (years) | Mean (SD) | 63.0 | (8.84) | 63.7 | (8.96) | 63.5 | (8.91) |
| | Median | 63.5 | | 65.0 | | 64.0 | |
| | Min, Max | 41, 81 | | 44, 83 | | 41, 83 | |
| Sex | Male | 45 | (52.3%) | 92 | (52.6%) | 137 | (52.5%) |
| | Female | 41 | (47.7%) | 83 | (47.4%) | 124 | (47.5%) |
| Ethnicity | Hispanic or Latino | 5 | (5.8%) | 3 | (1.7%) | 8 | (3.1%) |
| | Not Hispanic or Latino | 81 | (94.2%) | 172 | (98.3%) | 253 | (96.9%) |
| Race | American Indian or | 0 | (0.0%) | 0 | (0.0%) | 0 | (0.0%) |
| | Alaska Native | | | | | | |
| | Asian | 1 | (1.2%) | 1 | (0.6%) | 2 | (0.8%) |
| | Black or African | 7 | (8.1%) | 16 | (9.1%) | 23 | (8.8%) |
| | American | | | | | | |
| | Native Hawaiian or | 0 | (0.0%) | 0 | (0.0%) | 0 | (0.0%) |
| | Other Pacific Islander | | | | | | |
| | White | 77 | (89.5%) | 158 | (90.3%) | 235 | (90.0%) |
| | Other | 1 | (1.2%) | 0 | (0.0%) | 1 | (0.4%) |
| Weight (kg) | Mean (SD) | 83.0 | (20.36) | 84.2 | (21.24) | 83.8 | (20.92) |
| | Median | 79.8 | | 82.7 | | 82.2 | |
| | Min, Max | 46.3, 132.9 | | 44.0, 170.1 | | 44.0, 170.1 | |
| BMI (kg/m²) | Mean (SD) | 29.2 | (6.64) | 29.2 | (6.37) | 29.2 | (6.45) |
| | Median | 28.7 | | 28.9 | | 28.8 | |
| | Min, Max | 19.0, 53.6 | | 16.4, 50.8 | | 16.4, 53.6 | |
| FEV₁ (L) | Mean (SD) | 1.29 | (0.58) | 1.24 | (0.57) | 1.26 | (0.57) |
| | Median | 1.16 | | 1.16 | | 1.16 | |
| | Min, Max | 0.13, 3.10 | | 0.31, 3.50 | | 0.13, 3.50 | |
| FEV₁ percent predicted | Mean (SD) | 45.4 | (18.15) | 43.5 | (16.48) | 44.1 | (17.04) |
| | Median | 43.9 | | 42.0 | | 42.6 | |
| | Min, Max | 4.3, 90.0 | | 14.9, 109.0 | | 4.3, 109.0 | |
| | <30 | 0 | (0.0%) | 0 | (0.0%) | 0 | (0.0%) |
| | 30-<50 | 0 | (0.0%) | 0 | (0.0%) | 0 | (0.0%) |
| | 50-<80 | 0 | (0.0%) | 0 | (0.0%) | 0 | (0.0%) |
| | ≧80 | 0 | (0.0%) | 0 | (0.0%) | 0 | (0.0%) |
| FVC (L) | Mean (SD) | 2.49 | (0.89) | 2.41 | (0.79) | 2.44 | (0.83) |
| | Median | 2.30 | | 2.31 | | 2.31 | |
| | Min, Max | 0.22, 5.36 | | 1.00, 6.09 | | 0.22, 6.09 | |
| FVC percent predicted | Mean (SD) | 66.4 | (18.11) | 63.7 | (16.65) | 64.6 | (17.16) |
| | Median | 65.0 | | 63.0 | | 63.0 | |
| | Min, Max | 5.7, 113.1 | | 19.0, 126.6 | | 5.7, 126.6 | |
| FEV₁/FVC ratio | Mean (SD) | 0.53 | (0.15) | 0.53 | (0.19) | 0.53 | (0.18) |
| | Median | 0.52 | | 0.53 | | 0.52 | |
| | Min, Max | 0.25, 0.92 | | 0.18, 2.25 | | 0.18, 2.25 | |
| Ever received pneumococcal | Yes | 48 | (55.8%) | 115 | (65.7%) | 163 | (62.5%) |
| vaccine? | | | | | | | |
| | No | 19 | (22.1%) | 34 | (19.4%) | 53 | (20.3%) |
| | Unknown | 19 | (22.1%) | 26 | (14.9%) | 45 | (17.2%) |
| Received influenza vaccine for | Yes | 60 | (69.8%) | 124 | (70.9%) | 184 | (70.5%) |
| 2008/2009 season? | | | | | | | |
| | No | 24 | (27.9%) | 51 | (29.1%) | 75 | (28.7%) |
| | Unknown | 2 | (2.3%) | 0 | (0.0%) | 2 | (0.8%) |
| Current smoker? | Yes | 38 | (44.2%) | 79 | (45.1%) | 117 | (44.8%) |
| | No | 48 | (55.8%) | 96 | (54.9%) | 144 | (55.2%) |
| Number of years of regular | Mean (SD) | 37.3 | (11.73) | 38.9 | (10.72) | 38.4 | (11.06) |
| cigarette smoking | | | | | | | |
| | Median | 38.0 | | 40.0 | | 40.0 | |
| | Min, Max | 10, 67 | | 10, 64 | | 10, 67 | |
| Pack year of smoking [1] | Mean (SD) | 55.6 | (31.79) | 59.4 | (36.39) | 58.1 | (34.92) |
| | Median | 46.8 | | 50.0 | | 50.0 | |
| | Min, Max | 2.5, 180.0 | | 10.0, 260.1 | | 2.5, 260.1 | |
| Prescribed home/oxygen therapy | Yes | 26 | (30.2%) | 67 | (38.3%) | 93 | (35.6%) |
| | No | 60 | (69.8%) | 108 | (61.7%) | 168 | (64.4%) |
| Number of documented acute | Mean (SD) | 2.3 | (0.67) | 2.2 | (0.54) | 2.2 | (0.58) |
| exacerbation episodes in past 12 | Median | 2.0 | | 2.0 | | 2.0 | |
| months | Min, Max | 2, 5 | | 2, 5 | | 2, 5 | |
| Number of Patients taking | Inhaled and systemic | 50 | (58.1%) | 116 | (66.3%) | 166 | (63.6%) |
| specific medications at baseline | corticosteroids | | | | | | |
| for COPD | Long-acting beta | 48 | (55.8%) | 97 | (55.4%) | 145 | (55.6%) |
| | agonists | | | | | | |
| | Long-acting anti- | 39 | (45.3%) | 72 | (41.1%) | 111 | (42.5%) |
| | muscarinic agents | | | | | | |

[1] Calculated as average packs per day multiplied by years of smoking.

### Study Endpoints

Efficacy was evaluated using: (1) the incidence, duration and severity of exacerbation events; (2) sputum microbiology; (3) pulmonary function tests; (4) quality of life/symptoms and signs; and (5) the BODE index.

### Exacerbations

Acute exacerbations of COPD, including the rate of acute exacerbations of COPD between the two treatment groups were a primary endpoint of the study. An acute exacerbation included a symptomatic respiratory deterioration requiring treatment with antibiotic agents, systemic corticosteroids, hospitalization, or a combination of these treatments. In addition, exacerbations were characterized by increased sputum production, more purulent sputum, change in sputum color, increased coughing, increased wheezing, chest tightness, reduced exercise tolerance, increased fatigue, fluid retention, acute confusion, worsened dyspnea.

The characteristics of an acute exacerbation were measured using observations that included, for example, the medication required. The primary efficacy analysis included a comparison of exacerbation rates between the levofloxacin treatment group and the placebo treatment group. The secondary efficacy analysis included a comparison of the characteristics of any exacerbation between the levofloxacin treatment group and the placebo treatment group. Further observations that were measured also included dose of medication required, date of onset of an acute exacerbation, and duration of the acute exacerbation.

The duration and severity of a first acute exacerbation was analyzed from Cycle 1 to Final Visit. The duration of an acute exacerbation included the time from the beginning of treatment with antibiotics and/or systemic corticosteroids to the end of antibiotic and/or systemic corticosteroid treatment. Severity of an acute exacerbation was measured, for example, as ‘moderate’ where the use of antibiotics and/or systemic corticosteroids is required, and as ‘severe’ where hospitalization is required.

In addition, patients completed a daily questionnaire to report any symptom and exacerbation. Patients used standard definitions and items included questions related to breathlessness, cough and sputum, chest symptoms, global symptoms, healthcare intervention and changes in medications over the day. Standard definitions were provided by an Exacerbations of Chronic Obstructive Pulmonary Disease Tool (EXACT). EXACT provided a 14-item patient-reported outcome (PRO) measure designed to standardize the measurement approach for evaluating the frequency, severity, and duration of acute exacerbations of COPD in clinical trials (Jones, P. et al., (2007) “Quantifying of severity of exacerbations in chronic obstructive pulmonary disease: adaptations to the definition to allow quantification” Proc Am Thorac Soc. 4:597-601, incorporated by reference in its entirety). An exacerbation can include an increase of at least 2 standard deviations above an individual patient's average baseline score for at least 2 consecutive days, with the first of the two days serving as the day of onset. An increase in the score of an EXACT PRO can relate to an exacerbation and is associated with worsening of chronic symptoms, which can include but are not limited, breathlessness, cough, chest tightness, and nighttime waking. In more examples, the EXACT PRO can include terms and scores that relate to the presence of a symptom that includes increased sputum production, more purulent sputum, change in sputum color, increased coughing, increased wheezing, chest tightness, reduced exercise tolerance, increased fatigue, fluid retention, acute confusion, worsened dyspnea, and combinations thereof. In addition, the EXACT can include terms and scores that relate to a symptomatic respiratory deterioration requiring treatment with antibiotic agents, systemic corticosteroids, hospitalization, or a combination of these treatments.

Exacerbation rates in the levofloxacin and placebo groups were compared using a negative binomial regression model. The primary analysis was carried out using a one-sided test at the 5% level of significance. The distributions of time to first acute exacerbation and time to first EXACT-PRO exacerbation were estimated and summarized using the Kaplan-Meier method. The log-rank test (one-sided) was used to compare the distributions of the levofloxacin treatment group arm versus the placebo treatment group. Patients who did not have an exacerbation prior to discontinuation from the study were censored at the time of discontinuation. Other secondary endpoints were analyzed using two-sample t-tests and the Wilcoxon-Mann-Whitney test. Exacerbation rates between the two treatment groups were compared using hazard ratios.

Tables 7 and 8 summarize exacerbation rates in the MITT and EE populations, respectively. Exacerbation rates included the number of exacerbations per patient-year of study participation. Acute exacerbation included a symptomatic respiratory deterioration requiring treatment with antibiotic agents, systemic corticosteroids, hospitalization or a combination of these treatments. Rates and standard errors for each treatment, estimated ratio (MP-376/Placebo), 90% confidence interval of ratio, and one-sided p-values were calculated from a negative binomial regression model. Model 1 included terms for treatment group, age, baseline smoking status (Yes/No), number of exacerbations in past 12 months, and baseline percent predicted FEV₁ (categorized as quartiles). Model 2 included terms for treatment group only. Model 3 included all terms in Model 1 as well as additional terms for: baseline use of inhaled or systemic corticosteroids (Yes/No), baseline use of long-acting beta agonists (Yes/No), baseline use of long-acting anti-muscarinic agents (Yes/No), and baseline sputum bacterial pathogen colonization (Yes/No).

TABLE 7

| | | Placebo | MP-376 240 mg | Ratio (90% CI) | P- |
|---|---|---|---|---|---|
| (MITT Population) | | (N = 108) | BID (N = 214) | (MP-376/Placebo) | value |
| Exacerbation | Model 1 (primary | 1.20 (0.15) | 1.31 (0.11) | 1.09 (0.86, 1.39) | 0.7282 |
| Rate (SE) | comparison) | | | | |
| | Model 2 | 1.25 (0.15) | 1.35 (0.11) | 1.08 (0.84, 1.38) | 0.6931 |
| | Model 3 | 1.13 (0.14) | 1.22 (0.11) | 1.08 (0.86, 1.36) | 0.7108 |

TABLE 8

| | | Placebo | MP-376 240 mg | Ratio (90% CI) (MP- | P- |
|---|---|---|---|---|---|
| (EE Population) | | (N = 86) | BID (N = 175) | 376/Placebo) | value |
| Exacerbation | Model 1 (primary | 1.09 (0.15) | 1.21 (0.11) | 1.11 (0.85, 1.45) | 0.7444 |
| Rate (SE) | comparison) | | | | |
| | Model 2 | 1.16 (0.16) | 1.22 (0.11) | 1.05 (0.80, 1.37) | 0.6167 |
| | Model 3 | 1.02 (0.14) | 1.12 (0.11) | 1.10 (0.85, 1.41) | 0.7246 |

Tables 9 and 10 summarize EXACT-PRO exacerbation rates in the MITT and EE populations, respectively. EXACT-PRO Exacerbation rates included the number of exacerbations per patient-year as assessed by a change in daily EXACT-PRO scores. An exacerbation included an increase of at least 12 points above baseline for at least 2 consecutive days or 9 points above baseline for at least 3 consecutive days, with the first of the two (or three) days serving as the day of onset. Rates and standard errors (SE) for each treatment, estimated ratio (MP-376/Placebo), 90% confidence interval (CI) of ratio, and one-sided p-values were calculated from a negative binomial regression model. Model 1 included terms for treatment group, age, baseline smoking status (Yes/No), number of exacerbations in past 12 months, and baseline percent predicted FEV1 (categorized as quartiles). Model 2 included terms for treatment group only. Model 3 included all terms in Model 1 as well as additional terms for: baseline use of inhaled or systemic corticosteroids (Yes/No), baseline use of long-acting beta agonists (Yes/No), baseline use of long-acting anti-muscarinic agents (Yes/No), and baseline sputum bacterial pathogen colonization (Yes/No). EXACT-PRO Exacerbation Rate (rolling average) was calculated using a rolling 3-day average rather than individual daily scores. EXACT-PRO Event Rate (6 point increase) was calculated using a 6 point increase rather than a 12 point increase. EXACT-PRO Exacerbation Rate (reset baseline) was calculated using a 12 point increase, except the baseline value was reset prior to Day 1 of each cycle. A trend for reduced exacerbation rates were observed in the EE population of COPD patients administered aerosolized levofloxacin formulated with MgCl₂ compared to COPD patients administered placebo, as measured using EXACT-PRO models (Exacerbations of Chronic Obstructive Pulmonary Disease Tool (EXACT) with a 14-item patient-reported outcome (PRO)).

TABLE 9

| | | | MP-376 | | |
|---|---|---|---|---|---|
| | | Placebo | 240 mg BID | Ratio (90% CI) | P- |
| (MITT Population) | | (N = 108) | (N = 208) | (MP-376/Placebo) | value |
| EXACT-PRO | Model 1 | 1.32 (0.19) | 1.35 (0.13) | 1.03 (0.77, 1.36) | 0.5581 |
| Exacerbation Rate (SE) | Model 2 | 1.36 (0.19) | 1.39 (0.14) | 1.02 (0.77, 1.36) | 0.5527 |
| | Model 3 | 1.25 (0.17) | 1.21 (0.13) | 0.96 (0.73, 1.27) | 0.4132 |
| EXACT-PRO | Rate (SE) | 1.46 (0.20) | 1.50 (0.14) | 1.03 (0.78, 1.35) | 0.5621 |
| Exacerbation Rate | Model 1 | | | | |
| (rolling average) | | | | | |
| EXACT-PRO Event Rate | Rate (SE) | 2.92 (0.31) | 2.95 (0.22) | 1.01 (0.82, 1.25) | 0.5356 |
| (6 point increase) | Model 1 | | | | |
| EXACT-PRO | Rate (SE) | 1.55 (0.17) | 1.65 (0.13) | 1.06 (0.85, 1.33) | 0.6778 |
| Exacerbation Rate (reset | Model 1 | | | | |
| baseline) | | | | | |

TABLE 10

| | | | MP-376 | | |
|---|---|---|---|---|---|
| | | Placebo | 240 mg BID | Ratio (90% CI) | P- |
| (EE Population) | | (N = 86) | (N = 169) | (MP-376/Placebo) | value |
| EXACT-PRO | Model 1 | 1.36 (0.21) | 1.23 (0.13) | 0.91 (0.67, 1.23) | 0.2955 |
| Exacerbation Rate (SE) | Model 2 | 1.46 (0.22) | 1.27 (0.14) | 0.87 (0.64, 1.18) | 0.2202 |
| | Model 3 | 1.27 (0.19) | 1.10 (0.13) | 0.87 (0.64, 1.17) | 0.2150 |
| EXACT-PRO | Rate (SE) | 1.50 (0.22) | 1.37 (0.14) | 0.91 (0.68, 1.22) | 0.3049 |
| Exacerbation Rate | Model 1 | | | | |
| (rolling average) | | | | | |
| EXACT-PRO Event Rate | Rate (SE) | 2.93 (0.34) | 2.84 (0.23) | 0.97 (0.77, 1.22) | 0.4109 |
| (6 point increase) | Model 1 | | | | |
| EXACT-PRO | Rate (SE) | 1.66 (0.20) | 1.61 (0.14) | 0.97 (0.76, 1.23) | 0.4151 |
| Exacerbation Rate (reset | Model 1 | | | | |
| baseline) | | | | | |

Tables 11 and 12 summarize time to first acute exacerbation in the MITT and the EE populations, respectively.

TABLE 11

| | | | MP-376 | |
|---|---|---|---|---|
| | | Placebo | 240 mg BID | P- |
| (MITT Population) | | (N = 108) | (N = 214) | value |
| Time to First Acute | 25th | 89 [68, 140] | 90 [57, 113] | |
| Exacerbation. | Median | 253 [157, 334] | 242 [193, 285] | |
| Percentiles | 75th | N/A [334, N/A] | N/A | |
| [90% CI] | | | | |

TABLE 12

| | | | MP-376 | |
|---|---|---|---|---|
| | | Placebo | 240 mg BID | P- |
| (EE Population) | | (N = 86) | (N = 175) | value |
| Time to First Acute | 25th | 108 [79, 151] | 85 [57, 130] | |
| Exacerbation. | Median | 261 [173, N/A] | 260 [198, 306] | |
| Percentiles | 75th | N/A [334, N/A] | N/A | |
| [90% CI] | | | | |

Tables 13 and 14 summarize time to first acute EXACT-PRO exacerbation in the MITT population, and EE population, respectively. An exacerbation included an increase of at least 12 points above baseline for at least 2 consecutive days or 9 points above baseline for at least 3 consecutive days, with the first of the two (or three) days serving as the day of onset. Time to first acute EXACT-PRO Exacerbation (rolling average) was calculated using a rolling 3-day exacerbation average rather than individual daily scores. Time to first acute EXACT-PRO Event (6 point increase) was calculated using a 6 point increase rather than a 12 point increase. Time to first acute EXACT-PRO Exacerbation (reset baseline) was calculated using a 12 point increase, except the baseline value was reset prior to Day 1 of each cycle. A trend for increased time to first acute exacerbation was observed in COPD patients administered aerosolized levofloxacin formulated with MgCl₂ compared to COPD patients administered placebo, as measured using EXACT-PRO models.

TABLE 13

| | | | MP-376 | |
|---|---|---|---|---|
| | | Placebo | 240 mg BID | P- |
| (MITT Population) | | (N = 108) | (N = 214) | value |
| Time to First Acute | 25th | 27 [16, 64] | 39 [26, 61] | |
| EXACT-PRO | Median | 207 [106, N/A] | 250 [151, N/A] | |
| Exacerbation. | 75th | N/A | N/A | |
| Percentiles | | | | |
| [90% CI] | | | | |
| Time to First Acute | 25th | 32 [18, 58] | 31 [21, 46] | |
| EXACT-PRO | Median | 208 [103, N/A] | 224 [139, 313] | |
| Exacerbation. | 75th | N/A | N/A | |
| (rolling average) | | | | |
| Percentiles | | | | |
| [90% CI] | | | | |
| Time to First Acute | 25th | 10 [7, 15] | 10 [8, 14] | |
| EXACT-PRO Event | Median | 38 [22, 69] | 44 [27, 63] | |
| (6 point increase). | 75th | 251 [87, N/A] | N/A [185, N/A] | |
| Percentiles | | | | |
| [90% CI] | | | | |
| Time to First Acute | 25th | 29 [16, 80] | 35 [26, 46] | |
| EXACT-PRO | Median | 129 [105, 263] | 136 [101, 190] | |
| Exacerbation | 75th | N/A | N/A | |
| (reset baseline). | | | | |
| Percentiles | | | | |
| [90% CI] | | | | |

TABLE 14

| | | | MP-376 | |
|---|---|---|---|---|
| | | Placebo | 240 mg BID | P- |
| (EE Population) | | (N = 86) | (N = 175) | value |
| Time to First | 25th | 22 [16, 64] | 45 [30, 68] | |
| Acute EXACT- | Median | 197 [83, N/A] | 257 [151, N/A] | |
| PRO | 75th | N/A | N/A | |
| Exacerbation. | | | | |
| Percentiles | | | | |
| [90% CI] | | | | |
| Time to | 25th | 23 [15, 74] | 35 [26, 62] | |
| First Acute | Median | 180 [83, N/A] | 251 [152, 313] | |
| EXACT-PRO | 75th | N/A | N/A | |
| Exacerbation | | | | |
| (rolling average). | | | | |
| Percentiles | | | | |
| [90% CI] | | | | |
| Time to First | 25th | 10 [7, 15] | 12 [8, 14] | |
| Acute EXACT- | Median | 38 [20, 69] | 42 [28, 64] | |
| PRO Event | 75th | 113 [79, N/A] | N/A [151, N/A] | |
| (6 point increase). | | | | |
| Percentiles | | | | |
| [90% CI] | | | | |
| Time to | 25th | 22 [16, 82] | 39 [30, 61] | |
| First Acute | Median | 123 [100, 261] | 159 [113, 216] | |
| EXACT-PRO | 75th | N/A [263, N/A] | N/A [329, N/A] | |
| Exacerbation | | | | |
| (reset baseline). | | | | |
| Percentiles | | | | |
| [90% CI] | | | | |

Tables 15 and 16 summarize exacerbation rates based on individual components of primary endpoints in the MITT and the EE populations, respectively. Rates and standard errors for each treatment, estimated ratio (MP-376/Placebo), 90% confidence interval of ratio, and one-sided p-values were calculated from a negative binomial regression model including terms for treatment group, age, baseline smoking status (Yes/No), number of exacerbations in past 12 months, and baseline percent predicted FEV1 (categorized as quartiles). A trend for a reduced rate of acute exacerbations in patients that required hospitalization was observed in COPD patients administered aerosolized levofloxacin formulated with MgCl₂ compared to COPD patients administered placebo. Such a trend is consistent with treatment reducing the severity of acute exacerbations.

TABLE 15

| | | | MP-376 | Ratio | |
|---|---|---|---|---|---|
| | | | 240 mg | (90% CI) | |
| | | Placebo | BID | (MP-376/ | P- |
| (MITT Population) | | (N = 108) | (N = 214) | Placebo) | value |
| Required | Number of | 30 | 47 | | |
| hospitali- | Events | | | | |
| zation | Number of | 78.5 | 155.0 | | |
| | Patient Years | | | | |
| | Rate (SE) | 0.35 | 0.27 | 0.79 | 0.2019 |
| | | (0.08) | (0.05) | (0.49, 1.26) | |

TABLE 16

| | | | MP-376 | Ratio | |
|---|---|---|---|---|---|
| | | | 240 mg | (90% CI) | |
| | | Placebo | BID | (MP-376/ | P- |
| (EE Population) | | (N = 86) | (N = 175) | Placebo) | value |
| Required | Number of | 24 | 31 | | |
| hospitali- | Events | | | | |
| zation | Number of | 62.7 | 125.8 | | |
| | Patient Years | | | | |
| | Rate (SE) | 0.31 | 0.21 | 0.69 | 0.1155 |
| | | (0.08) | (0.04) | (0.42, 1.15) | |

Tables 17 and 18 summarize hospitalization and unscheduled healthcare visits in the MITT and EE populations, respectively. One sided p-values were calculated using Pearson's chi-square test for comparison of proportions and from a negative binomial regression model with a main effect of treatment group for comparison of rates. A trend for reduced numbers of respiratory-related hospitalizations was observed in COPD patients administered aerosolized levofloxacin formulated with MgCl₂ compared to COPD patients administered placebo. In addition, a trend for reduced numbers of exacerbation-related hospitalizations was observed in COPD patients administered aerosolized levofloxacin formulated with MgCl₂ compared to COPD patients administered placebo.

TABLE 17

| | | | MP-376 | Ratio | |
|---|---|---|---|---|---|
| | | | 240 mg | (90% CI) | |
| | | Placebo | BID | (MP-376/ | P- |
| (MITT Population) | | (N = 108) | (N = 214) | Placebo) | value |
| Number of respiratory-related | Number of | 33 | 65 | | |
| hospitalizations per patient | Events | | | | |
| year of study participation | Number of | 78.5 | 155.0 | | |
| | Patient | | | | |
| | Years | | | | |
| | Rate (SE) | 0.4 | 0.43 | 0.97 | 0.4614 |
| | | (0.10) | (0.07) | (0.63, 1.52) | |
| Number of exacerbation- | Number of | 31 | 49 | | |
| related hospitalizations per | Events | | | | |
| patient year of study | Number of | 78.5 | 155.0 | | |
| participation | Patient | | | | |
| | Years | | | | |
| | Rate (SE) | 0.41 | 0.32 | 0.78 | 0.2019 |
| | | (0.10) | (0.06) | (0.48, 1.27) | |

TABLE 18

| | | | MP-376 | Ratio (90% CI) | |
|---|---|---|---|---|---|
| | | Placebo | 240 mg BID | (MP-376/ | P- |
| (EE Population) | | (N = 86) | (N = 175) | Placebo) | value |
| Number of respiratory-related | Number of | 27 | 43 | | |
| hospitalizations per patient | Events | | | | |
| year of study participation | Number of | 62.7 | 125.8 | | |
| | Patient | | | | |
| | Years | | | | |
| | Rate (SE) | 0.45 | 0.35 | 0.78 (0.49, 1.24) | 0.1847 |
| | | (0.10) | (0.06) | | |
| Number of exacerbation- | Number of | 25 | 33 | | |
| related hospitalizations per | Events | | | | |
| patient year of study | Number of | 62.7 | 125.8 | | |
| participation | Patient | | | | |
| | Years | | | | |
| | Rate (SE) | 0.42 | 0.27 | 0.64 (0.38, 1.09) | 0.0820 |
| | | (0.10) | (0.05) | | |

### Microbiological Evaluations

Bacteria were identified and quantified in patients' sputum, including S. pneumoniae, B-hemolytic streptococci, S. aureus, H. influenzae, M. catarrhalis, P. aeruginosa, and other enterobacteriaceae. In addition, the MIC for particular bacteria to levofloxacin was measured.

Sputum from COPD patients administered levofloxacin formulated with MgCl₂ had a lower density of bacteria, including, S. pneumoniae, B-hemolytic streptococci, S. aureus, H. influenzae, M. catarrhalis, P. aeruginosa, and other enterobacteriaceae. In addition, MIC₅₀ and MIC₉₀ of bacteria, such as Pseudomonas aeruginosa, to levofloxacin in patients treated with levofloxacin did not change significantly during the course of the study (data not shown). This indicated that the bacteria did not develop resistance to the levofloxacin during the course of the study.

Table 19 summarizes the change in bacterial density of specific organisms within cycles in the MITT population. P-values were calculated using a one-sided Wilcoxon-Mann-Whitney test. Mean, median, minimum and maximum bacterial density units are log₁₀ CFU/g sputum.

TABLE 19

| | Time | | | MP-376 | |
|---|---|---|---|---|---|
| Organism | Point | Statistic | Placebo | 240 mg BID | P-value |
| S. pneumoniae | Cycle 1, | N | 8 | 8 | |
| | Change | Mean (SD) | −0.91 (4.34) | −4.06 (4.78) | 0.0943 |
| | from Day 1 | Median | 0.05 | −6.46 | |
| | to Day 6 | Min, Max | −7.30, 5.34 | −7.78, 4.92 | |
| | Final | N | 8 | 5 | |
| | Cycle, | Mean (SD) | 2.75 (5.59) | −3.41 (5.57) | 0.9607 |
| | Change | Median | 3.63 | −6.78 | |
| | from Day 1 | Min, Max | −7.78, 7.96 | −7.78, 5.15 | |
| | to Day 6 | | | | |
| B-hemolytic | Cycle 1, | N | 6 | 8 | |
| streptococci | Change | Mean (SD) | 0.98 (4.18) | −4.24 (1.60) | 0.0100 |
| | from Day 1 | Median | 1.70 | −4.39 | |
| | to Day 6 | Min, Max | −4.30, 5.08 | −7.00, −1.65 | |
| | Final | N | 5 | 17 | |
| | Cycle, | Mean (SD) | −2.37 (2.21) | −1.63 (3.80) | 0.6230 |
| | Change | Median | −3.40 | −2.05 | |
| | from Day 1 | Min, Max | −4.30, 0.12 | −7.00, 5.41 | |
| | to Day 6 | | | | |
| S. aureus | Cycle 1, | N | 13 | 34 | |
| | Change | Mean (SD) | 0.76 (2.23) | −1.35 (3.02) | 0.0344 |
| | from Day 1 | Median | 0.60 | −0.60 | |
| | to Day 6 | Min, Max | −1.78, 5.90 | −7.38, 3.30 | |
| | Final | N | 12 | 17 | |
| | Cycle, | Mean (SD) | −0.27 (2.86) | −1.34 (2.89) | 0.1439 |
| | Change | Median | −0.61 | −2.22 | |
| | from Day 1 | Min, Max | −5.60, 4.32 | −5.10, 4.85 | |
| | to Day 6 | | | | |
| H. influenzae | Cycle 1, | N | 3 | 4 | |
| | Change | Mean (SD) | 0.69 (0.09) | −5.64 (1.94) | 0.0249 |
| | from Day 1 | Median | 0.70 | −5.39 | |
| | to Day 6 | Min, Max | 0.60, 0.78 | −7.78, −4.00 | |
| | | Mean (SD) | −0.76 (7.77) | −4.85 (N/A) | 0.5000 |
| | | Median | −1.19 | −4.85 | |
| | | Min, Max | −7.78, 7.11 | −4.85, −4.85 | |
| | Final | N | 4 | 1 | |
| | Cycle, | Mean (SD) | −0.78 (4.54) | −3.60 (N/A) | 0.6382 |
| | Change | Median | −1.60 | −3.60 | |
| | from Day 1 | Min, Max | −5.15, 5.24 | −3.60, −3.60 | |
| | to Day 6 | | | | |
| M. catarrhalis | Cycle 1, | N | 4 | 5 | |
| | Change | Mean (SD) | −0.55 (6.87) | −7.57 (0.13) | 0.1311 |
| | from Day 1 | Median | −1.10 | −7.60 | |
| | to Day 6 | Min, Max | −7.78, 7.78 | −7.70, −7.34 | |
| | Final | N | 2 | 2 | |
| | Cycle, | Mean (SD) | 0.15 (10.79) | −5.78 (2.83) | 0.3493 |
| | Change | Median | 0.15 | −5.78 | |
| | from Day 1 | Min, Max | −7.48, 7.78 | −7.78, −3.78 | |
| | to Day 6 | | | | |
| P. aeruginosa | Cycle 1, | N | 11 | 14 | |
| | Change | Mean (SD) | 0.52 (3.46) | −1.97 (3.83) | 0.0295 |
| | from Day 1 | Median | 0.09 | −2.04 | |
| | to Day 6 | Min, Max | −4.75, 7.10 | −7.60, 7.00 | |
| | Final | N | 10 | 10 | |
| | Cycle, | Mean (SD) | −1.36 (5.19) | −1.22 (3.77) | 0.6613 |
| | Change | Median | −0.95 | −1.46 | |
| | from Day 1 | Min, Max | −7.72, 7.90 | −7.39, 3.24 | |
| | to Day 6 | | | | |
| Other | Cycle 1, | N | 30 | 43 | |
| enterobacteriaceae | Change | Mean (SD) | 0.57 (2.21) | −2.35 (2.46) | <.0001 |
| | from Day 1 | Median | 0.32 | −1.48 | |
| | to Day 6 | Min, Max | −3.34, 7.38 | −7.78, 2.50 | |
| | Final | N | 23 | 24 | |
| | Cycle, | Mean (SD) | 0.43 (2.58) | −0.78 (2.38) | 0.0666 |
| | Change | Median | 1.20 | −1.00 | |
| | from Day 1 | Min, Max | −4.75, 7.48 | −6.14, 4.90 | |
| | to Day 6 | | | | |

### Pulmonary Function Evaluations

All patients underwent pulmonary function testing to determine their forced vital capacity (FVC) and forced expiratory volume in one second (FEV₁). FEV₁/FVC ratio, FVC percent predicted, and FEV₁ percent predicated. Pulmonary function tests were performed according to American Thoracic Society/European Respiratory Society (ATS/ERS) Spirometry Standards (2005), incorporated by reference in its entirety.

Table 20 summarizes data obtained from pulmonary function tests in the MITT population. Mean, median, Min, Max, LS Mean values are for percent change between baseline and final visit of the final cycle. Estimates were determined from a repeated measures model with terms for treatment, visit, treatment*visit, baseline, and visit*baseline. P-values were one-sided.

TABLE 20

| | | Placebo | MP-376 240 mg | LS Mean Diff [90% CI] | P- |
|---|---|---|---|---|---|
| Test | | (N = 108) | BID (N = 214) | (MP-376 - Placebo) | value |
| FVC (L) | N | 79 | 156 | | |
| | Mean | 23.51 | 3.44 | | |
| | (SD) | (159.05) | (25.28) | | |
| | Median | −1.08 | −1.68 | | |
| | Min, Max | −57.46, 1377.27 | −68.42, 111.11 | | |
| | LS Mean | 21.21 | 1.25 | −20.0 [−37.5, −2.42] | 0.9693 |
| | (SE) | (8.63) | (6.17) | | |
| FEV₁ (L) | N | 79 | 156 | | |
| | Mean | 29.25 | 6.64 | | |
| | (SD) | (215.17) | (33.86) | | |
| | Median | −3.26 | 0.00 | | |
| | Min, Max | −41.35, 1892.31 | −75.23, 188.42 | | |
| | LS Mean | 24.44 | 4.19 | −20.3 [−44.3, 3.80] | 0.9171 |
| | (SE) | (11.85) | (8.49) | | |
| FVC percent | N | 79 | 156 | | |
| predicted | Mean | 25.06 | 4.71 | | |
| | (SD) | (158.49) | (24.54) | | |
| | Median | 0.00 | 0.00 | | |
| | Min, Max | −56.85, 1371.93 | −47.45, 82.05 | | |
| | LS Mean | 23.78 | 1.57 | −22.2 [−39.3, −5.12] | 0.9836 |
| | (SE) | (8.41) | (6.01) | | |
| FEV₁ percent | N | 79 | 155 | | |
| predicted | Mean | 30.92 | 6.78 | | |
| | (SD) | (213.10) | (31.76) | | |
| | Median | 0.00 | 2.17 | | |
| | Min, Max | −40.00, 1874.42 | −43.64, 163.33 | | |
| | LS Mean | 25.87 | 4.68 | −21.2 [−44.9, 2.53] | 0.9293 |
| | (SE) | (11.68) | (8.37) | | |

To the extent publications and patents or patent applications incorporated by reference herein contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, unless otherwise expressly stated, should be construed as open ended as opposed to limiting. As examples of the foregoing, the term ‘including’ should be read to mean ‘including, without limitation’ or the like; the term ‘comprising’ as used herein is synonymous with ‘including,’ ‘containing,’ or ‘characterized by,’ and is inclusive or open-ended and does not exclude additional, unrecited elements or method steps; the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise. In addition, as used in this application, the articles ‘a’ and ‘an’ should be construed as referring to one or more than one (i.e., to at least one) of the grammatical objects of the article. By way of example, ‘an element’ means one element or more than one element.

The presence in some instances of broadening words and phrases such as ‘one or more’, ‘at least’, ‘but not limited to’, or other like phrases shall not be read to mean that the narrower case is intended or required in instances where such broadening phrases may be absent.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method for reducing a rate of acute exacerbations of a pulmonary disorder in a human having said disorder, comprising administering to said human an aerosol of a solution comprising levofloxacin or ofloxacin, wherein the rate of exacerbations in said human is reduced to less than about 6 exacerbations/patient year.

2. The method of claim 1, wherein the rate of exacerbations in said human is reduced to less than about 3 exacerbations/patient year.

3. The method of claim 1, wherein the rate of exacerbations in said human is reduced to less than about 1.4 exacerbations/patient year.

4. The method of claim 1, wherein the rate of exacerbations in said human is reduced to less than about 1.2 exacerbations/patient year.

5. The method of claim 1, wherein the rate of exacerbations in said human is reduced to less than about 1.0 exacerbations/patient year.

6. The method of claim 1, wherein the rate of exacerbations in said human is reduced to less than about 0.5 exacerbations/patient year.

7. The method of claim 1, wherein the solution further comprises a divalent or trivalent cation.

8. The method of claim 1, wherein the solution comprises no lactose.

9. The method of claim 1, wherein the solution comprises a divalent or trivalent cation concentration from about 50 mM to about 400 mM, and levofloxacin or ofloxacin concentration from between about 50 mg/ml to about 200 mg/ml.

10. The method of claim 1, wherein the solution comprises a divalent or trivalent cation concentration from about 100 mM to about 300 mM, and levofloxacin or ofloxacin concentration from between about 75 mg/ml to about 150 mg/ml.

11. The method of claim 1, wherein the solution comprises a divalent or trivalent cation concentration from about 150 mM to about 250 mM, and levofloxacin or ofloxacin concentration from between about 90 mg/ml to about 125 mg/ml.

12. The method of claim 1, wherein the solution comprises an osmolality from about 300 mOsmol/kg to about 500 mOsmol/kg, and a pH from about 5 to about 8.

13. The method of claim 1, wherein the solution comprises an osmolality from about 350 mOsmol/kg to about 425 mOsmol/kg, and a pH from about 5 to about 6.5.

14. The method of claim 1, wherein the solution comprises a pH from about 5.5 to about 6.5.

15. The method of claim 1, wherein the solution comprises a divalent or trivalent cation selected from magnesium, calcium, zinc, copper, aluminum, and iron.

16. The method of claim 1, wherein the solution comprises magnesium chloride.

17. The method of claim 1, wherein the solution comprises levofloxacin or ofloxacin concentration between about 90 mg/ml to about 110 mg/ml, a magnesium chloride concentration between about 175 mM to about 225 mM, a pH between about 5 to about 7; an osmolality of between about 300 mOsmol/kg to about 500 mOsmol/kg, and lacks lactose.

18. The method of claim 1, wherein the aerosol comprises a mass median aerodynamic diameter from about 2 microns to about 5 microns with a geometric standard deviation less than or equal to about 2.5 microns.

19. The method of claim 1, wherein the aerosol comprises a mass median aerodynamic diameter from about 2.5 microns to about 4.5 microns with a geometric standard deviation less than or equal to about 1.8 microns.

20. The method of claim 1, wherein the aerosol comprises a mass median aerodynamic diameter from about 2.8 microns to about 4.3 microns with a geometric standard deviation less than or equal to about 2 microns.

21. The method of claim 1, wherein the aerosol is produced with a vibrating mesh nebulizer.

22. The method of claim 21, wherein the vibrating mesh nebulizer is a PARI E-FLOW® nebulizer.

23. The method of claim 1, wherein at least about 20 mg of levofloxacin or ofloxacin is administered to the lungs of the human.

24. The method of claim 1, wherein at least about 100 mg of levofloxacin or ofloxacin is administered to the lungs of the human.

25. The method of claim 1, wherein at least about 125 mg of levofloxacin or ofloxacin is administered to the lungs of the human.

26. The method of claim 1, wherein at least about 150 mg of levofloxacin or ofloxacin is administered to the lungs of the human.

27. The method of claim 1, further comprising co-administering an additional active agent selected from the group consisting of antibiotics, bronchodilators, anticholinergics, glucocorticoids, eicosanoid inhibitors, and combinations thereof.

28. The method of claim 1, comprising administering the aerosol once daily.

29. The method of claim 1, comprising administering the aerosol twice daily.

30. The method of claim 1, wherein the pulmonary disorder is chronic obstructive pulmonary disorder (COPD).

31. The method of claim 1, wherein the pulmonary disorder is chronic bronchitis (CB).
